SEQUENCE LISTING (3 sequences)

SEQ ID NO: 1 - protein, 19 aa, Gallus gallus
GEFTGTYTTAVTATSHEIK

SEQ ID NO: 2 - protein, 12 aa, Gallus gallus
TQPTFGFTVNWK

SEQ ID NO: 3 - protein, 11 aa, Gallus gallus
SSVNDIGDDWK

(19) United States
(12) Patent Application Publication
(10) Pub. No.: US 2021/0388337 A1
(43) Pub. Date: Dec. 16, 2021

(54) PROTEIN-POLYMER CONJUGATES AND METHODS FOR THEIR PREPARATION

(71) Applicant: Carnegie Mellon University, Pittsburgh, PA (US)

(72) Inventors: Hironobu Murata, Pittsburgh, PA (US); Alan J. Russell, Pittsburgh, PA (US)

(21) Appl. No.: 17/284,837

(22) PCT Filed: Oct. 11, 2019
(86) PCT No.: PCT/US2019/055977

§ 371 Date: Apr. 13, 2021

Related U.S. Application Data
(60) Provisional application No. 62/766,380, filed on Oct. 13, 2018.

(51) Int. Cl. C12N 9/96; C12N 11/087; A61K 47/62; A61K 47/60; A61K 47/58

(52) U.S. Cl. CPC C12N 9/96; C12N 11/087; A61K 47/58; A61K 47/60; A61K 47/62

(57) ABSTRACT

This document relates to materials and methods for using controlled radical polymerization (e.g., atom transfer radical polymerization) to generate protein-polymer conjugates in which two or more polymer molecules are attached to individual initiator molecules on the protein.

### CROSS-REFERENCE TO RELATED APPLICATIONS

This application claims benefit of priority from U.S. Provisional Application Ser. No. 62/766,380, filed Oct. 13, 2018.

### STATEMENT AS TO FEDERALLY SPONSORED RESEARCH

This invention was made with government support under Defense Threat to Reduction Agency: No. HDTRA1-18-1-0028. The government has certain rights in this invention.

### TECHNICAL FIELD

This document relates to materials and methods for using atom transfer radical polymerization (ATRP) to generate protein-polymer conjugates in which two or more polymer molecules are attached to individual initiator molecules on the protein.

### BACKGROUND

Protein-polymer conjugates are unique macromolecules that combine the rugged attractiveness of synthetic chemistry and the exquisite balance of activity and specificity found in biological systems. Since the synthesis of the first protein-polymer conjugate was reported in 1977, the application of protein-polymer conjugates has expanded significantly (Abuchowski et al., J Biol Chem 252:3578-3581, 1977). Today these conjugates are used in biotechnology (Hills, Eur J Lipid Sci Technol 105:601-607, 2003), cosmetics (Bi et al., J Agric Food Chem 63:1558-1561, 2015), foods, surface coatings and therapeutics (Wu et al., Biomaterials Sci 3:214-230, 2015).

Protein-polymer conjugates can be synthesized using two different strategies, known as “grafting to” or “grafting from.” The process of “grafting to” consists of covalent attachment of pre-synthesized and characterized polymers to the protein (Grover and Maynard, Current Opin Chem Biol 14:818-127, 2010). A limitation of this method has been low achievable grafting densities of polymers on protein surfaces due to steric hindrance created by subsequently attached polymer chains. Also, control of the attachment site location and purification of the resulting conjugates can be challenging (Jevsevar et al., Biotechnol J 5:113-128, 2010; Treetharnmathurot et al., Int J Pharmaceutics 357:252-259, 2008; and Murata et al., Biomacromolecules 15:2817-2823, 2014). In the “grafting from” approach, the polymers are generated from the protein surface by controlled radical polymerization (CRP). Most commonly, either ATRP or reversible addition-fragmentation polymerization (RAFT) methods have been used (Cummings et al., Biomacromolecules 18:576-586, 2017; Averick et al., ACS Macro Lett 1:6-10, 2012; and Lele et al., Biomacromolecules 6:3380, 2005). The “grafting from” method has enabled tighter control over modification site, high grafting density and simplified purification. Since the number and molecular weight of polymer chains are predetermined, the method allows the generation of protein-polymer conjugates with low dispersity (D).

Polymer-based protein engineering has been used to prepare conjugates with enhanced pH and temperature stability, and tailored substrate affinity and stability in organic environments (Murata et al., supra; Lele et al., supra; Gao et al., Proc Natl Acad Sci USA 106:15231, 2009; Qi et al., Macromol Rapid Commun 34:1256, 2013; Murata et al., Biomacromolecules 14:1919-1926, 2013; Cummings et al., ACS Macro Lett 5:493, 2016; and Cummings et al., Biomaterials 34:7437, 2013). In recent years, considerable attention has been paid to the creation of “smart conjugates” by anchoring stimuli responsive polymers that respond to temperature and pH (Heredia et al., J Am Chem Soc 127:16955-16960, 2005; Cobo et al., Nature Materials 14:143, 2014; and Cummings et al., Biomacromolecules 15:763, 2014). However, understanding of how the polymer layer affects substrate diffusion limits and rates to the active site of proteins is limited. Polymer sieving properties are important criteria affecting the efficacy of protein drugs Liu et al., Nature Commun 5:5526, 2014) and biomedical devices (Hucknall et al., Adv Mater 21:2441-2446, 2009). For example, enzyme-polymer conjugates used in therapy should repel proteins from the immune system and different types of proteases, while allowing their substrates to reach the active site (FIG. 1). Comb-shaped poly(oligo(ethylene glycol) methacrylate) (pOEGMA) polymers can create a molecular sieving effect when grafted from a chymotrypsin surface by blocking larger macromolecules (Liu et al., Adv Funct Mater 23:2007-2015, 2013). However, no one has been able to determine the rate at which molecules penetrate the polymer shell grown around proteins.

### SUMMARY

Provided herein is a well-controlled system and method for understanding the relationship between polymer length/density and the diffusion/accessibility of different size/shape substrates (Lin et al., Colloids and Surfaces B: Biointerfaces 146:888-894, 2016; Chien et al., Colloids and Surfaces B: Biointerfaces 107:152-159, 2013; Leigh et al., Biomacromolecules 18:2389-2401, 2017; Chen et al., Polymer 51:5283-5293, 2010; and Schlenoff, Langmuir 30:9625-9636, 2014). The system utilizes a novel, N-hydroxysuccinimide- (NHS-) functionalized, multi-headed ATRP initiator that supports the growth of two or more polymers from one initiation point. This document therefore provides materials and methods for making and using protein-polymer conjugates in which two or more polymer chains are coupled to a single initiator molecule on the surface of a protein. In some embodiments, the polypeptide-polymer conjugates provided herein can have a higher density of polymers coupled to the protein than polypeptide-polymer conjugates assembled using single-headed initiators.

In a first aspect, this document features a polypeptide-polymer conjugate that includes a polypeptide, one or more initiator molecules conjugated to the polypeptide, where each of the one or more initiator molecules includes two or more ATRP initiation groups, and a polymer molecule conjugated to each of the ATRP initiation groups. The initiator molecule can have two ATRP initiation groups. The initiator molecule can be 4-(bis(2-(2-bromo-2-methylpropanamido)ethyl)amino)-4-oxobutyloyl-N-oxysuccinimide ester. The polymer can be selected from the group consisting of poly(oligo(ethylene glycol) methacrylate) (pOEGMA), poly(carboxybetaine methacrylate) (pCBMA), copolymers of poly(oxyethylene)allylmethyldiether and maleic anhydride, copolymers of poly(oxyethylene)2-methyl-2-propenylmethyldiether and maleic anhydride, α-methoxy-poly(ethylene glycol) (MPEG), poly(polyethylene glycol monomethyl ether methacrylate) (PPEGMA), poly(2-dimethylaminoethyl methacrylate) (pDMAEMA), poly(sulfobetaine methacrylate) (pSBMA), poly(2-(methylsulfinyl)ethyl acrylate) (pMSEA), poly(N,N-dimethylaminoethyl methacrylate), poly(quaternary ammonium ethyl methacrylate), poly(hydroxyethyl)methacrylate, 2-azidoethyl methacrylate, and epoxy methacrylate. The polypeptide can be an enzyme (e.g., an esterase, lipase, organophosphate hydrolase, aminase, oxidoreductase, hydrogenase, lysozyme, transaminase, asparaginase, protease, or uricase).

In another aspect, this document features a method for generating a polypeptide-polymer conjugate. The method can include coupling one or more initiator molecules to a polypeptide to generate a polypeptide-initiator complex, where each of the one or more initiator molecules includes two or more ATRP initiation groups; and growing, via controlled radical polymerization, a polymer molecule from each of said two or more ATRP initiation groups, thus generating a polypeptide-polymer conjugate. The initiator molecule can have two ATRP initiation groups. The ATRP initiation groups can be alkyl bromide or alkyl chloride groups. The initiator molecule can be 4-(bis(2-(2-bromo-2-methylpropanamido)ethyl)amino)-4-oxobutyloyl-N-oxysuccinimide ester. The polymer can be selected from the group consisting of pOEGMA, pCBMA, copolymers of poly(oxyethylene)allylmethyldiether and maleic anhydride, copolymers of poly(oxyethylene)2-methyl-2-propenylmethyldiether and maleic anhydride, MPEG, PPEGMA, pDMAEMA, pSBMA, pMSEA, poly(N,N-dimethylaminoethyl methacrylate), poly(quaternary ammonium ethyl methacrylate), poly(hydroxyethyl)methacrylate), 2-azidoethyl methacrylate, and epoxy methacrylate. The polypeptide can be an enzyme (e.g., an esterase, lipase, organophosphate hydrolase, aminase, oxidoreductase, hydrogenase, lysozyme, transaminase, asparaginase, protease, or uricase). The controlled radical polymerization can be atom transfer radical polymerization.

In another aspect, this document features a polypeptide-polymer conjugate obtained by coupling one or more initiator molecules to a polypeptide to generate a polypeptide-initiator complex, where each of the one or more initiator molecules includes two or more ATRP initiation groups; and growing, via controlled radical polymerization, a polymer molecule from each of the two or more ATRP initiation groups, thus generating a polypeptide-polymer conjugate.

In still another aspect, this document features a conjugate containing a polypeptide having one or more initiator molecules coupled thereto, where each initiator molecule includes two or more ATRP initiation groups. The initiator molecule can be 4-(bis(2-(2-bromo-2-methylpropanamido)ethyl)amino)-4-oxobutyloyl-N-oxysuccinimide ester.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram illustrating the molecular sieving effect by polymers grown from the surface of avidin, allowing small molecule substrates to reach the avidin surface, but blocking larger molecules such as enzymes and antibodies.

FIGS. 2A and 2B are diagrams illustrating the synthesis of avidin-pCBMA conjugates using PBPE. FIG. 2A illustrates the synthesis of low-density avidin-pCBMA conjugates. Step (1) includes single-headed ATRP initiator modification on native avidin, while step (2) includes a “grafting from” reaction to synthesize avidin-pCBMA conjugates. FIG. 2B illustrates the synthesis of high-density avidin-pCBMA conjugates. Step (1) includes double-headed ATRP initiator modification on native avidin, and step (2) includes a “grafting from” reaction to synthesize double-headed avidin-pCBMA conjugates.

FIGS. 3A and 3B are MALDI-ToF mass spectroscopy spectra for native avidin (FIG. 3A) and single-headed initiator-modified avidin conjugate (FIG. 3B) with 8 initiators attached. The number of modifications was determined by subtracting m/z of native avidin from m/z avidin-single-headed initiator conjugates and dividing by the initiator molar mass without the NHS group (220 Da).

FIGS. 4A-4D show the structure of avidin and ESI mass spectroscopy for trypsin digested native avidin or modified avidin. FIG. 4A shows the crystal structure of avidin (PDB:2AVI). The K45, K71 and K111 residues are located near the biotin binding site of avidin. FIG. 4B shows ESI mass spectroscopy for trypsin-digested native avidin. FIG. 4C shows ESI mass spectroscopy for trypsin-digested avidin-Br. FIG. 4D shows ESI mass spectroscopy for trypsin digested avidin-(Br)₂. The absence of native peaks for K45 at 1058.1 m/z (GEFTGTYTTAVTATSNEIK m/z, [M+3ACN+2H]²⁺; SEQ ID NO:1), K71 at 713.8 m/z (TQPTFGFTVNWK m/z, [M+2H]²⁺; SEQ ID NO:2) and K111 at 639.2 m/z (SSVNDIGDDWK m/z, [M+ACN+2H]²⁺; SEQ ID NO:3) suggest that these amine groups were modified with single and double-headed ATRP initiators.

FIG. 5 is a graph showing GPC traces of cleaved pCBMA from low-grafting density avidin-pCBMA conjugates. Gel permeation chromatography was used to determine the molecular weight of pCBMA polymers cleaved from the avidin surface by acid hydrolysis. pCBMA polymers were dissolved at 2 mg/mL using 0.1 M sodium phosphate buffer, pH 8.0 as the eluent. Samples were run at a flow rate of 1 mL/min. Pullulan standards were used for calibration.

FIGS. 6A-6E are graphs plotting the particle size distribution of low-density avidin-pCBMA conjugates by number distribution. FIG. 6A, native avidin; FIG. 6B, avidin-pCBMA₅₆; FIG. 6C, avidin-pCBMA₁₂₁; FIG. 6D, avidin-pCBMAro; FIG. 6E, avidin-pCBMA₂₃₂. Native avidin and each of the low-grafting density conjugates were dissolved at 1 mg/mL concentration using 0.1 M sodium phosphate, pH 8. Hydrodynamic diameters were measured three times (5 run each measurement) at room temperature.

FIGS. 7A-7H are graphs plotting intrinsic tryptophan fluorescence changes for native avidin and low-density avidin conjugates upon biotin binding. FIG. 7A, native avidin fluorescence intensity before and after adding biotin. FIG. 7B, avidin-pCBMA₅₆ fluorescence intensity before and after biotin binding. FIG. 7C, avidin-initiator conjugate fluorescence intensity changes before and after biotin binding. FIG. 7D, avidin-initiation inhibitor conjugate fluorescence intensity changes before and after binding biotin. FIG. 7E, native avidin fluorescence quenching with different concentrations of free initiator. FIG. 7F, native avidin fluorescence quenching with different concentrations of free initiation inhibitor. FIG. 7G, effect of biotin on the fluorescence of native avidin incubated with free initiator. FIG. 7H, effect of biotin on the fluorescence of native avidin incubated with free initiation inhibitor.

FIGS. 8A and 8B are MALDI-ToF mass spectroscopy plots for native avidin (FIG. 8A) and initiation inhibitor-modified avidin with 8 initiation inhibitor molecules (FIG. 8B). The number of modifications was determined by subtracting m/z of native avidin from m/z of avidin-initiation inhibitor conjugates and dividing by the initiator molar mass without the NHS group (140 Da).

FIGS. 9A-9I show fluorescence spectra for native and biotinylated aprotinin, histone, and HRP mixed with avidin-pCBMA₅₆. FIG. 9A, fluorescence spectrum of aprotinin-bio; FIG. 9B, fluorescence spectrum of native aprotinin mixed with avidin-pCBMA₅₆; FIG. 9C, binding spectrum of aprotinin-bio by avidin-pCBMA₅₆; FIG. 9D, control spectrum of histone-bio; FIG. 9E, control spectrum of native histone mixed with avidin-pCBMA₅₆; FIG. 9F, binding spectrum of avidin-pCBMA₅₆ and histone-bio; FIG. 9G, fluorescence spectrum of HRP-bio; FIG. 9H, control spectrum of avidin-pCBMA₅₆ and native HRP; and FIG. 9I, binding of HRP bio by avidin-pCBMA₅₆.

FIG. 10 is a graph plotting the binding rates of biotinylated substrates to low-density avidin-pCBMA conjugates as a function of substrate size. The substrates included biotin-PEG 550 Da (1.9±0.8 nm), biotin-aprotinin (2.4±0.4 nm), biotin-PEG 5K (4.2±0.5 nm), biotin-histone (4.6±0.2 nm), biotin-HRP (5.1±0.7 nm), biotin-PEG 10K (6.1±0.5 nm), and biotin-PEG 30K (9.4±0.9 nm). Avidin-pCBMA₅₆ (circles), avidin-pCBMA₁₂₁ (squares), avidin-pCBMA₁₇₀ (triangles), and avidin-pCBMA₂₃₂ (hexagons).

FIGS. 11A-11C show polymerization from free dual initiator. FIG. 11A illustrates the synthesis and hydrolysis of free double-headed pCBMA. (1), ATRP from free double-headed initiator. (2), acid hydrolysis of free double-headed pCBMA. (3), hydrolysable amide bonds are shown with arrows. FIG. 11B is a GPC trace of free single-headed pCBMA before and after acid hydrolysis. FIG. 11C is a GPC trace of free double-headed pCBMA before and after acid hydrolysis.

FIGS. 12A and 12B are MALDI-ToF mass spectroscopy spectra for native avidin (FIG. 12A) and double-headed initiator-modified avidin conjugate with 7 initiators (FIG. 12B). The number of modifications was determined by subtracting m/z of native avidin from m/z of avidin-double-headed initiator conjugates and dividing by the initiator molar mass without the NHS group (473 Da).

FIG. 13 shows GPC traces for cleaved pCBMA from high-grafting density avidin-pCBMA conjugates. The pCBMA was cleaved by acid hydrolysis.

FIGS. 14A-14E are graphs plotting particle size distribution of high-grafting density avidin-pCBMA conjugates by number distribution. FIG. 14A, native avidin; FIG. 14B, avidin-pCBMA₅₈; FIG. 14C, avidin-pCBMA₁₀₉; FIG. 14D, avidin-pCBMA₁₅₂; FIG. 14E, avidin-pCBMA₁₈₂. Native avidin and each of the conjugates were dissolved at 1 mg/mL concentration using 0.1 M sodium phosphate, pH 8. Hydrodynamic diameters were measured three times (5 run each measurement) at room temperature.

FIG. 15 is a graph plotting the binding rates of biotinylated substrates to high-density avidin-pCBMA conjugates as a function of substrate size. High-density avidin-pCBMA₅₈ (circles), high-density avidin-pCBMA₁₀₉ (squares), high-density avidin-pCBMA₁₅₂ (triangles), and high-density avidin-pCBMA₁₈₂ (diamonds).

FIG. 16A is a scheme illustrating a method for synthesizing a double-headed ATRP initiator. FIG. 16B is a scheme illustrating a method for synthesizing a quadruple-headed ATRP initiator.

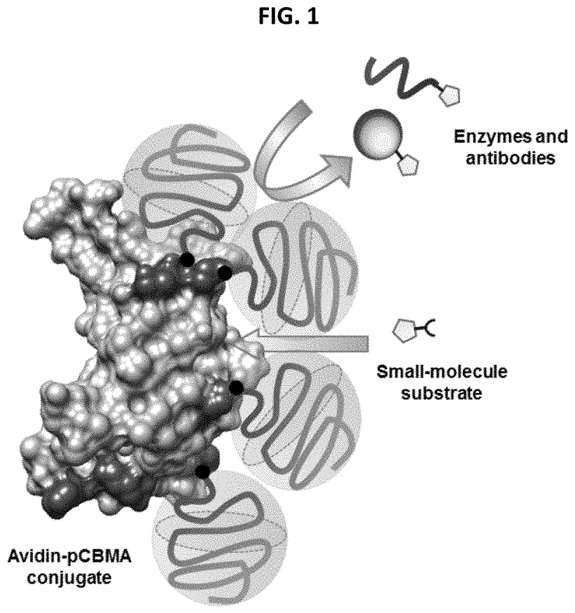

### DETAILED DESCRIPTION

This document provides materials and methods that can include the synthesis and use of a novel, NHS-functionalized, multi-headed ATRP initiator that supports the growth of two or more polymers from one initiation point. As described herein, the initiator was used in the CRP synthesis of eight different molecular weight avidin-pCBMA conjugates, which were then employed to study the penetration rate of molecules through the polymer shell to the protein binding site as a function of polymer chain length, polymer grafting density, substrate size, and substrate shape.

CRP is a type of polymerization in which the active polymer chain end is a free radical. A more specific CRP technique is ATRP, which is a type of a reversible-deactivation radical polymerization, and is a means of forming a carbon-carbon bond with a transition metal catalyst. ATRP typically employs an alkyl halide (R-X) initiator and a transition metal complex (e.g., a complex of Cu, Fe, Ru, Ni, or Os) as a catalyst. In an ATRP reaction, the dormant species is activated by the transition metal complex to generate radicals via electron transfer. Simultaneously, the transition metal is oxidized to a higher oxidation state. This reversible process rapidly establishes an equilibrium that predominately is shifted to the side with very low radical concentrations. The number of polymer chains is determined by the number of initiators, and each growing chain has the same probability of propagating with monomers to form living/dormant polymer chains (R-Pₙ-X). As a result, polymers with similar molecular weights and narrow molecular weight distribution can be prepared.

The basic ATRP process and a number of improvements are described elsewhere. See, for example, U.S. Pat. Nos. 5,763,546; 5,807,937; 5,789,487; 5,945,491; 6,111,022; 6,121,371; 6,124,411; 6,162,882; 6,624,262; 6,407,187; 6,512,060; 6,538,091; 6,541,580; 6,624,262; 6,627,314; 6,759,491; 6,790,919; 6,887,962; 7,019,082; 7,049,373; 7,064,166; 7,125,938; 7,157,530; 7.332,550; 7,407,995; 7,572,874; 7,678,869; 7,795,355; 7,825,199; 7,893,173; 7,893,174; 8,252,880; 8,273,823; 8,349,410; 8,367,051; 8,404,788; 8,445,610; 8,816,001; 8.865,795; 8,871,831; 8,962,764; 9,243,274; 9,410,020; 9,447,042; 9,533,297; and 9,644,042; and Publication Nos. 2014/0183055; 2014/0275420; and 2015/0087795.

ATRP also is discussed in a number of publications and reviewed in several book chapters. See, e.g., Matyjaszewski and Zia, Chem Rev 101:2921-2990, 2001; Qiu et al., Prog Polym Sci 26:2083-2134, 2001; Wang and Matyjaszewski, J Am Chem Soc 117:5614-5615, 1995; Coessens et al., Prog Polym Sci 26:337-377, 2001; Braunecker and Matyjaszewski, Prog Polym Sci 32:93-146, 2007; Matyjaszewski, Macromol 45:4015-4039, 2012; Schroder et al., ACS Macro Letters 1:1037-1040, 2012; Matyjaszewski and Tsarevsky, J Am Chem Soc 136:6513-6533, 2014; and Kamigaito et al., Chem Rev 101:3689-3746, 2001. Indeed, ATRP can control polymer composition, topology, and position of functionalities within a copolymer (Coessens et al., supra; Advances in Polymer Science; Springer Berlin/Heidelberg: 2002, Vol. 159; Gao and Matyjaszewski, Prog. Polym. Sci. 34:317-350, 2009; Blencowe et al., Polymer 50:5-32, 2009; Matyjaszewski, Science 333:1104-1105, 2011; and Polymer Science: A Comprehensive Reference, Matyjaszewski and Martin, Eds., Elsevier: Amsterdam, 2012; pp 377-428). All of the above-mentioned patents, patent application publications, and non-patent references are incorporated herein by reference to provide background and definitions for the present disclosure.

Monomers and initiators having a variety of functional groups (e.g., allyl, amino, epoxy, hydroxy, and vinyl groups) can be used in ATRP. ATRP has been used to polymerize a wide range of commercially available monomers, including various styrenes, (meth)acrylates, (meth)acrylamides, N-vinylpyrrolidone, acrylonitrile, and vinyl acetate as well as vinyl chloride (Qiu and Matyjaszewski, Macromol. 30:5643-5648, 1997; Matyjaszewski et al, J. Am. Chem. Soc. 119:674-680, 1997; Teodorescu and Matyjaszewski, Macromol. 32:4826-4831, 1999; Debuigne et al., Macromol. 38:9488-9496, 2005; Lu et al., Polymer 48:2835-2842, 2007; Wever et al., Macromol. 45:4040-4045, 2012; and Fantin et al., J. Am. Chem. Soc. 138:7216-7219, 2016). In general, non-limiting examples of monomers that can be used in ATRP reactions include carboxybetaine methacrylate (CBMA), oligo(ethylene glycol) methacrylate (OEGMA), 2-dimethylaminoethyl methacrylate (DMAEMA), sulfobetaine methacrylate (SBMA), 2-(methylsulfinyl)ethyl acrylate (MSEA), oligo(ethylene oxide) methyl ether methacrylate (OEOMA), and (hydroxyethyl)methacrylate (HEMA).

ATRP can be used to add polymer chains to the surfaces of proteins. An initial step in a protein-ATRP reaction is the addition of initiator molecules to the protein surface. In some cases, ATRP initiators (1) contain an alkyl halide as the point of initiation, (2) are water soluble, and (3) contain a protein-reactive “handle.” Alkyl halide ATRP-initiators usually include NHS groups that react with protein primary amines, including the N-terminal and lysine residues. Targeting amino groups can be an effective way to achieve the highest polymer coating due to the high abundance of amino groups on protein surfaces. The initiation reaction can be somewhat controlled using carefully designed algorithms that can predict specific reaction rates and sites of the individual amino groups (Carmali et al., ACS Biomater Sci Eng 2017, 3(9):2086-2097).

The molecular sieving properties of protein surface-attached polymers are the central features in how polymers extend therapeutic protein lifetimes in vivo. Polymers have been grown from the surface of avidin using ATRP to determine how polymer length and density influence the binding kinetics of ligands as a function of ligand size and shape. The rate of ligand binding is strongly dependent on the density of polymer grafting and the size of the substrate, but interestingly, far less dependent on the length of the polymer. The work described herein unveils the mysteries of how polymers attached to a protein surface influence the access of biomacromolecules to binding sites on the protein.

Protein-ligand interactions can be studied with avidin-biotin complexes (Muegge and Martin, J Medicinal Chem 42:791-804, 1999; MacBeath et al., J Am Chem Soc 121:7967-7968, 1999; and Wilchek et al., Immunol Lett 103:27-32, 2006). Avidin is a tetrameric protein purified from egg white that binds biotin with exquisite strength and speed. The high affinity of avidin toward biotin allows for the biotinylation of substrates with different shapes and sizes, and subsequent monitoring of their permeation rates through covalently-attached polymer layers of varying lengths and densities. By using ATRP to decorate avidin with pCBMA polymers, a zwitterionic polymer that has non-fouling properties, a well-controlled system was created for understanding the relationship between polymer length/density and the diffusion/accessibility of different size/shape substrates (Lin et al., supra, 2016; Chien et al., supra; Leigh et al., supra; Chen et al., supra; and Schlenoff, supra).

Any appropriate protein can be coupled to an initiator and subsequently subjected to CRP using the methods provided herein. In some embodiments, for example, an enzyme (e.g., an esterase, lipase, organophosphate hydrolase, aminase, oxidoreductase, hydrogenase, lysozyme, transaminase, asparaginase, protease, or uricase) can be used in the methods described herein. Other protein that can be used in the methods and conjugates provided herein include, without limitation, avidin.

This document provides multi- (e.g., double-) headed ATRP initiators, as well as protein-initiator and protein-polymer conjugates containing such multi-headed initiators, and methods for generation and use of such multi-headed initiators, protein-initiator conjugates, and protein-polymer conjugates.

Any appropriate ATRP initiator can be used in the methods provided herein. Suitable initiators can be based on, for example, 2-bromopropanitrile (BPN), ethyl 2-bromoisobutyrate (BriB), ethyl 2-bromopropionate (EBrP), methyl 2-bromopropionate (MBrP), 1-phenyl ethylbromide (1-PEBr), tosyl chloride (TsCl), 1-cyano-1-methylethyldiethyldithiocarbamte (MANDC), 2-(N,N-diethyldithiocarbamyl)-isobutyric acid ethyl ester (EMADC), dimethyl 2,6-dibromoheptanedioate (DMDBHD), 2-chloro-2-methypropyl ester (CME), 2-chloropropanitrile (CPN), ethyl 2-chloroisobutyrate (CliB), ethyl 2-chloropropionate (EC1P), methyl 2-chloropropionate (MC1P), dimethyl 2,6-dichloroheptanedioate (DMDC1HD), or 1-phenyl ethylchloride (1-PEC1).

The amino group at the N-terminus of a protein typically has a pKa in the range of 7.8-8.0, while the pKa's of lysine side chains range from about 10.5 to 12.0, depending on their local environment (Murata et al., Nat. Commun. 2018, 9, 845). Therefore, at biologically relevant pH values (6-8), the accessible amino groups are positively charged. During ATRP reactions, these positive charges are lost upon initiator attachment, as most (if not all) initiators typically used in ATRP reactions are neutral (see, e.g., Le Droumaguet and Nicolas, Polym. Chem. 2010, 1(5):563; and Broyer et al., Chem. Commun. 2011, 47(8):2212). In some cases, therefore, an initiator can include a group with a positive charge (in addition to an amine-reactive group and one or more alkyl halide or other groups that can react with a monomer to initiate polymer addition to the protein). For example, neutral initiator molecules such as those listed above can be modified by reaction with N-(3-N′,N′-dimethylaminopropyl)-2-bromo-2-methylpropanamide in the presence of acetonitrile, resulting in a molecule with an amine-reactive group, an alkyl halide from which monomer addition can be initiated, and a positively charged quaternary ammonium group.

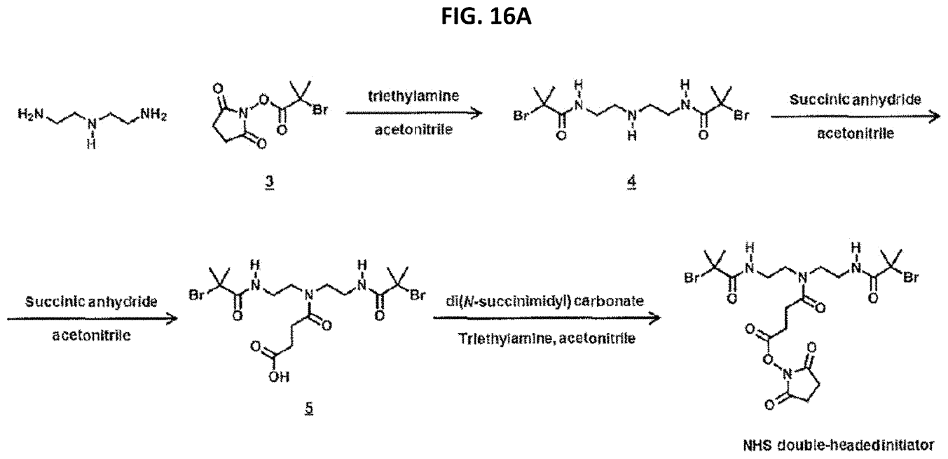

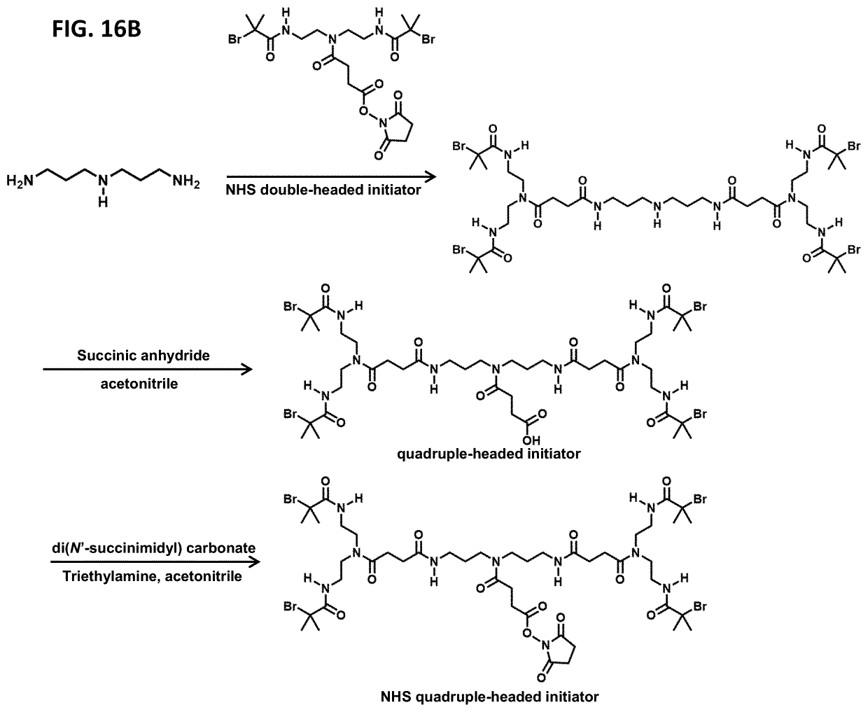

The initiators listed above have a single alkyl halide group from which to initiate polymer growth. The number of chains grown from a protein using “grafting from” ATRP with amino-reactive, single-headed initiators cannot exceed the number of accessible amine groups on the surface of the protein. To overcome this limitation and better understand the impact of polymer density on the rate of sieving by attached polymers, a novel, NHS-functionalized, double-headed ATRP initiator was designed to support the growth of two polymers from one initiation point, as described in the Examples herein. As illustrated in FIG. 16A, for example, a protein surface active, double-headed ATRP initiator can be synthesized from dimethylalkylamine and an alkylbromide containing an active ester such as N-oxysuccinimide. FIG. 16B illustrates a method for synthesizing a quadruple-headed initiator.

Thus, the initiator molecules used in the conjugates and methods provided herein can, in some cases, be “double-headed” such that each initiator molecule can be used as the start point for growing two separate polymer molecules. In some cases, an initiator molecule can have more than two “heads,” such that more than two polymer molecules can be grown from each “multi-headed” initiator molecule. In some cases, therefore, an initiator can have two or more (e.g., two, three, four, five, six, or more than six) alkyl halide groups from which to initiate polymer growth.

As described herein, an exemplary double headed initiator (4-(bis(2-(2-bromo-2-methylpropanamido)ethyl)amino)-4-oxobutyloyl-N-oxysuccinimide ester) was used for synthesis of eight different molecular weight avidin-pCBMA conjugates, which were used to study the penetration rate of molecules through the polymer shell to the protein binding site as a function of polymer chain length, polymer grafting density, substrate size, and substrate shape. Polymer grafting density and substrate size can have profound effects on the rate of binding of ligands to proteins shielded with covalently attached polymers. Surprisingly, as discussed in the Examples herein, the molecular weight of the polymer attached to the protein and shape of the diffusing molecule had only a small impact on the rate of ligand binding.

Any suitable polymer can be used in the conjugates and methods described herein. In some cases, comb-type polymers can be particularly useful (e.g., for molecular sieving applications). Comb-type polymers consist of a main chain with two or more three-way branch points and linear side chains. The side chains can all be identical to one another, or they may be different from one another. Comb-type polymers are generally more well-defined than other branched polymers (e.g., hyperbranched polysaccharides). In addition, comb density, length, and bulkiness can be adjusted to confer particular properties to the polymer and thus to the protein-polymer conjugates resulting from attachment of the polymer to a polypeptide.

Examples of polymers that can be used in the conjugates and methods provided herein include, without limitation, poly(oligo(ethylene glycol) methacrylate) (pOEGMA), poly(carboxybetaine methacrylate) (pCBMA), copolymers of poly(oxyethylene)allylmethyldiether and maleic anhydride, copolymers of poly(oxyethylene)2-methyl-2-propenylmethyldiether and maleic anhydride, α-methoxy-poly(ethylene glycol) (MPEG), poly(polyethylene glycol monomethyl ether methacrylate) (PPEGMA), poly(2-dimethylaminoethyl methacrylate) (pDMAEMA), poly(sulfobetaine methacrylate) (pSBMA), poly(2-(methylsulfinyl)ethyl acrylate) (pMSEA), poly(N,N-dimethylaminoethyl methacrylate), poly(quaternary ammonium ethyl methacrylate), poly(hydroxyethyl)methacrylate, 2-azidoethyl methacrylate, and epoxy methacrylate. It is noted that in some cases, maleic anhydride can be used to generate comb-type polymers.

Any appropriate method can be used to synthesize polymers (e.g., comb-type polymers) from an initiator on the surface of a polypeptide. In general, CRP can be carried out using standard methods. For ATRP, for example, a protein-initiator/protein-blocker complex can be contacted with a population of monomers and a transition metal catalyst that includes a metal ligand complex. Any appropriate metal ligand complex can be used. The transition metal in the metal ligand complex can be, for example, copper, iron, cobalt, zinc, ruthenium, palladium, or silver. The ligand in the metal ligand complex can be, without limitation, an amine-based ligand (e.g., 2,2′-bipyridine (bpy), 4,4′-di(5-nonyl)-2,2′-bipyridine (dNbpy), N,N,N′,N′-tetramethylethylenediamine (TMEDA), N-propyl(2-pyridyl)methanimine (NPrPMI), 2,2′:6′,2″-terpyridine (tpy), 4,4′,4″-tris(5-nonyl)-2,2′:6′,2″-terpyridine (tNtpy), N,N,N′,N″,N″-pentamethyldiethylenetriamine (PMDETA), N,N-bis(2-pyridylmethyl)octylamine (BPMOA), 1,1,4,7,10,10-hexamethyltriethylenetetramine (HMTETA), tris[2-(dimethylamino)ethyl]amine (Me₆TREN), tris[(2-pyridyl)methyl]amine (TPMA), 1,4,8,11-tetraaza-1,4,8,11-tetramethylcyclotetradecane (Me₄CYCLAM), or N,N,N′,N′-tetrakis(2-pyridylmethyl)ethylenediamine (TPEN).

In some embodiments, this document provides protein-initiator conjugates in which a protein is coupled to a CRP (e.g., ARTP) initiator having an amine-reactive group and two or more alkyl halide groups. The amine-reactive group can react with amine groups on a protein surface, while the alkyl halide groups can react with monomers to initiate polymerization. Any suitable amine-reactive group can be used. Examples of appropriate amine-reactive groups include active esters (e.g., N-hydroxysuccinimide ester, nitrophenol ester, pentafluorophenol ester, can oxybenzotriaole ester). Further, any suitable alkyl halides can be used for monomer reaction. In some cases, the alkyl halides can include bromine or chlorine atoms. If present, any suitable group can provide a positive charge to an initiator used in the methods provided herein. In some cases, for example, an initiator can include a positively charged quaternary ammonium.

Also provided herein are methods for generating protein-initiator conjugates, where the methods include contacting a protein with a multi-headed CRP initiator. The initiator can include an amine-reactive group for reaction with amine groups on a protein surface, and two or more alkyl halide groups for reaction with monomers to initiate polymerization. Again, any suitable amine-reactive group and any suitable alkyl halide can be used, including those listed herein.

In some cases, the methods provided herein can include using CRP (e.g., ATRP) to generate a protein-polymer conjugate from a protein-initiator conjugate prepared as described herein. For example, a protein-initiator conjugate can be contacted with a population of monomers in the presence of a transition metal catalyst or metal-free organic complex that can participate in a redox reaction.

The invention will be further described in the following examples, which do not limit the scope of the invention described in the claims.

### EXAMPLES

### Example 1—Materials and Methods

Materials: Avidin from egg white was purchased from Lee Biosolutions (Maryland Heights, Mo.). Aprotinin and Histone were purchased from Sigma Aldrich (St. Louis, Mo.). Horse Radish Peroxidase was purchased from Millipore Sigma (Burlington, Mass.). Biotin-PEG-NHS was purchased from Thermo Fisher (Waltham, Mass.). Biotin-PEG was purchased from Creative PEG Workers (Chapel Hill, N.C.). Single ATRP initiator was synthesized as described elsewhere (Murata et al., supra). Initiation inhibitor was prepared as described elsewhere (Carmali et al., Biomacromolecules 19(10):4044-4051, 2018).

NHS-Functionalized double-headed ATRP initiator synthesis: Double-headed ATRP initiator was synthesized as follows. N,N′-dicyclohexylcarbodimine (10.9 g, 53 mmol) in dichloromethane (10 mL) was slowly added to the solution of 2-bromo-isobutyric acid (8.0 g, 48 mmol) and N-hydroxysuccinimide (6.1 g, 53 mmol) in dichloromethane (100 mL) at 0° C. The mixture was stirred at room temperature overnight. Precipitated urea was filtered out and the filtrate was evaporated to remove solvent. 2-bromo-2-methylpropionyl-N-oxysuccinimine ester was isolated by recrystallization in 2-propanol. Next, 2-bromo-2-methylpropionyl-N-succinimide ester (5.3 g, 2.0 mmol) was slowly added to the solution of diethylenetriamine (1.0 g, 9.7 mmol) and triethylamine (1.4 mL, 1.0 mmol) in acetonitrile (50 mL) at 0° C. The mixture was stirred at room temperature overnight. Precipitated N-hydroxysuccinimide was filtered out and the filtrate was evaporated to remove solvent. Ethyl acetate (50 mL) was added to the mixture and the organic phase was washed with 50 wt % sodium carbonate aq. (20 mL×3) and saturated NaCl aq. (20 mL×3). The organic phase was dried with Na₂CO₃ and evaporated to remove solvent. Bis(2-(2-bromo-2-methylpropanamido) ethylamine was isolated by column chromatography (silica and acetonitrile). Succinic anhydride (600 mg, 6.0 mmol) and triethylamine (840 μL, 6.0 mmol) was added to the solution of bis(2-(2-bromo-2-methylpropanamido)ethylamine (2.2 g, 5.5 mmol) in acetonitrile (50 mL), and the mixture was stirred at room temperature overnight. After the solvent was evaporated, ethyl acetate (50 mL) was added to the mixture. The organic phase was washed with 1 N HCl aq. (20 mL×3) and saturated NaCl aq. (20 mL×3). The organic phase was dried with MgSO₄ and evaporated to remove solvent. To the solution of bis(2-(2-bromo-2-methylpropanamido)ethyl)amino)-4-oxobutanoic acid (2.0 g, 2.0 mmol) in acetonitrile (50 mL), di(N-succinimidyl) carbonate (1.1 g, 4.3 mmol) and triethylamine (560 μL, 4.0 mmol) were added and the mixture was stirred at room temperature overnight. After the solvent evaporated, double-headed ATRP initiator was isolated by column chromatography (silica, acetone:chloroform (1/4 volume ratio)) The chemical structures were confirmed by ¹H and ¹³C NMR and IR.

Attachment of single ATRP initiator on the surface of avidin: Synthesis of the ATRP initiator was carried out as described elsewhere (Murata et al. 2013, supra). After synthesis, the initiator (523 mg, 1.56 mmol) and avidin (500 mg, 0.03 mmol protein, 0.31 mmol primary amine groups) were dissolved in 100 mL of 0.1 M sodium phosphate buffer, pH 8. The solution was stirred at 4° C. for 2 hours and avidin conjugates were purified by dialysis using 15 kDa molecular mass cutoff dialysis tubing, in 25 mM sodium phosphate (pH 8), for 24 hours at 4° C. and then lyophilized.

Double-headed ATRP initiator attachment onto avidin surface: Following the synthesis, double-headed ATRP initiator (935 mg, 1.56 mmol) was dissolved in 4 mL of DMSO added to a solution of avidin (500 mg, 0.31 mmol primary amine groups) in 100 mL of 0.1 M sodium phosphate buffer, pH 8. The mixture was stirred at 4° C. and for 2 hours, then dialyzed against 25 mM sodium phosphate buffer (pH 8), using dialysis tubing with molecular mass cutoff of 15 kDa, for 24 hours at 4° C. and then lyophilized.

MALDI-ToF analysis: MALDI-ToF measurements were recorded using a PerSeptive Voyager STR MS with nitrogen laser (337 nm) and 20 kV accelerating voltage with a grid voltage of 90%. 500 laser shots covering the complete spot were accumulated for each spectrum. For determination of molecular weights of synthesized protein-initiator complexes, sinapinic acid (10 mg/mL) in 50% acetonitrile with 0.4% trifluoroacetic acid was used as matrix. Protein solution (1.0 mg/mL) was mixed with an equal volume of matrix and 2 μL of the resulting mixture was loaded onto a silver sterling plate. Apomyoglobin, cytochrome C, and aldolase were used as standard calibration samples. ATRP initiator modification was determined by subtracting the native protein m/z values from protein-initiator conjugate m/z values and then dividing by the molecular weight of the initiator (220.5 g/mol for single and 478 g/mol for double-headed initiators).

Trypsin digestion of avidin-initiator conjugates: Trypsin digests were used to generate peptide fragments from which initiator attachment sites could be determined using electrospray ionization mass spectrometry. Samples were digested according to the protocol described in the In-Solution Tryptic Digestion and Guanidination Kit. 20 μg of protein or protein-initiator complexes (10 μL of a 2 mg/mL protein solution in deionized water) were added to 15 μL of 50 mM ammonium bicarbonate with 1.5 μL of 100 mM dithiothreitol in a 0.5 mL Eppendorf tube. The reaction was incubated for 5 minutes at 95° C. Thiol alkylation was achieved by the addition of 3 μL of 100 mM iodoacetamide aqueous solution to the protein solution followed by a 20 minute incubation in the dark at room temperature. After the incubation, 1 μL of 100 ng trypsin was added to the protein solution and the reaction was incubated at 37° C. for 3 hours. Then, an additional 1 μL of 100 ng trypsin was subsequently added. The reaction was terminated after 2 hours by the addition of trifluoroacetic acid (TFA). Digested samples were purified using ZipTipC₁₈ microtips and eluted with 200 μL of matrix solution (50% acetonitrile with 0.1% formic) for subsequent ESI-MS analysis. The molecular weight of the expected peptide fragments before and after digestion was predicted using PeptideCutter (ExPASy Bioinformatics Portal, Swiss Institute of Bioinformatics).

ESI-MS analysis: ESI-MS measurements were taken by using a Finnigan LCQ (Thermo-Fisher) quadrupole field ion trap mass spectrometer with electrospray ionization (Yee et al., J Am Chem Soc 127:16512, 2005) source. Each scan was acquired over the range m/z 150-2000 by using a step of 0.5 u, a dwell time of 1.5 ms, a mass defect of 50 pu, and an 80-V orifice potential. Samples at a protein concentration of 50 μM and eluted using 50% acetonitrile and 0.1% formic acid at a flow rate of 20 μL/min.

ATRP from single-headed ATRP initiator modified avidin: To synthesize avidin-pCBMA conjugates, the avidin-initiator complex (50 mg, 0.0226 mmol of initiator groups) and CBMA monomer (259 mg, 1.1 mmol for avidin-pCBMA₅₀, 518 mg, 2.3 mmol for avidin-pCBMA₁₀₀, 777 mg, 3.4 mmol for avidin-pCBMA₁₅₀ and 1036 mg, 4.5 mmol for avidin-pCBMA₂₀₀ were dissolved in 45 mL of 0.1 M sodium phosphate. The flask was sealed with a rubber septum and bubbled with nitrogen for 1 hour. In a separate flask, 6 mL of 50 mM CuCl₂ solution was bubbled under nitrogen for 20 minutes. Sodium ascorbate (300 μL of 20 mg/mL, 0.1 mmol) and 1,1,4,7,10,10-hexamethyltriethylenetetramine (HMTETA) (100 μL, 0.37 mmol) were added to the deoxygenated CuCl₂ solution and bubbled for another 5 minutes. 5 mL of deoxygenated copper catalyst solution was added to a 45 mL solution of deoxygenated avidin-Br/CBMA in 100 mM sodium phosphate pH 8 and allowed to react for 1 hour at room temperature. The reaction was stopped upon exposure to air and avidin-pCBMA conjugates were purified through dialysis (25 kDa MWCO) against 25 mM sodium phosphate for 24 hours at 4° C. and then lyophilized.

ATRP from double-headed ATRP initiator modified avidin: Avidin-double-headed initiator conjugates (40 mg, 0.027 mmol initiator groups) and CBMA (310 mg, 1.35 mmol for avidin-pCBMA₅₀, 619 mg, 2.7 mmol for avidin-pCBMA₁₀₀, 929 mg, 4.1 mmol for avidin-pCBMA₁₅₀ and 1239 mg, 5.4 mmol for avidin-pCBMA₂₀₀) were dissolved in sodium phosphate buffer (45 mL, 0.1 M, pH 8). The solutions of avidin-initiator conjugates and monomers were sealed with a rubber septum and bubbled with nitrogen for 1 hour. 300 μL of sodium ascorbate (of 20 mg/mL, 0.1 mmol) and 100 μL of HMTETA were added to 6 mL of deoxygenated CuCl₂ (50 mM) and bubbled for 5 minutes. 5 mL of deoxygenated copper catalyst was added to a solution of deoxygenated avidin-Br/CBMA and allowed to react for 1 hour at room temperature. The reaction was stopped upon exposure to air and avidin-pCBMA conjugates were purified through dialysis (25 kDa MWCO) against 25 mM sodium phosphate for 24 hours at 4° C. and then lyophilized.

Cleavage of pCBMA from avidin surface: Avidin-pCBMA conjugates (20 mg) were placed in hydrolysis tubes and dissolved in 6 N HCl (6 mL). After five freeze-pump-thaw cycles, the hydrolysis was performed at 110° C. under vacuum for 24 hours. The cleaved polymers were dialyzed against deionized water at room temperature, using 1 kDa molecular mass cut off dialysis tubing and then lyophilized. The molecular weight and dispersity of polymers were measured by gel permeation chromatography (GPC).

BCA assay: Avidin conjugates were dialyzed against deionized water to remove salts present in the samples and then lyophilized. Next, 1.0 mg of conjugates were dissolved in deionized water and 25 μL of the sample was mixed with bicinchonic acid (BCA) solution (1.0) and copper (II) sulfate solution (50:1 vol:vol). The solution was incubated at 60° C. for 15 minutes. Absorbance of the sample was recorded at 562 nm using UV-VIS spectrometer. Avidin concentration (wt %) was determined by comparison of the absorbance to a standard curve (native avidin).

Measuring conjugate hydrodynamic diameter: The DLS data was collected on a Malvern Zetasizer nano-ZS. Native avidin and avidin conjugates (1.0 mg) were dissolved in 0.1 M sodium phosphate, pH 8. The hydrodynamic diameter (Dh) of the samples was measured three times (12 runs/measurement). Reported values are number distribution intensities.

Protein biotinylation: For biotinylation, 20 mg of aprotinin (0.0031 mmol protein), histone (0.00093 mmol protein) and HRP (0.00045 mmol protein) were dissolved in 0.1 M sodium phosphate buffer (4 mL, pH 8). 18.2 mg of biotin-PEG-NHS (0.031 mmol) for aprotinin, 5.4 mg (0.0093 mmol) for histone and 2.6 mg for HRP (0.0045 mmol) and were dissolved in 2004 of DMSO and added to a protein solution. The solution was stirred at 4° C. for 2 hours and protein-biotin conjugates were purified by dialysis using 15 kDa molecular mass cutoff dialysis tube in deionized water and then lyophilized.

Fluorescamine assay: A fluorescamine assay was used to determine the biotinylation extend of proteins. Protein-biotin samples (80 μL, 1.0 mg/mL), 100 mM sodium phosphate (80 μL, pH 8.5), and fluorescamine solution in DMSO (40 μL, 3 mg/mL) were added into a 96-well plate and incubated at room temperature for 15 minutes. Fluorescence intensities were measured at an excitation wavelength of 390 nm and emission of 470 nm with 10-nm bandwidths by a H Synergy plate reader. Biotinylation was determined by comparison of the fluorescence to the standard curve (native proteins).

Intrinsic tryptophan fluorescence of avidin: For tryptophan fluorescence measurements native avidin, avidin-initiator conjugates and avidin-pCBMA conjugates (180 μL, final concentration of avidin 5 μM) and biotin (20 μL, final concentration 10 μM) were mixed in 96-well plate. The tryptophan fluorescence intensities were measured at an excitation wavelength of 270 nm. The emission spectrum was observed from 300 nm to 400 nm with bandwidth of 2 nm using H Synergy Plate reader. The intrinsic fluorescence was measured in triplicate.

Tryptophan fluorescence quenching assay: Intrinsic tryptophan fluorescence intensity of native avidin (180 μL, final concentration 5 μM) was measured at an excitation wavelength of 270 nm and emission of 300-400 nm in a 96-well plate. N-2-bromo-2-methylpropanoyl-β-alanine (free initiator) (20 μL, final concentrations 20 μM, 80 μM, 320 μM, 1.28 mM, 5.12 mM) or N-2-methylpropanoyl-β-alanine (free initiation inhibitor) (20 μL, final concentrations 20 μM, 80 μM, 320 μM, 1.28 mM, 5.12 mM) were added to native avidin and tryptophan fluorescence was measured again.

Biotin effect on quenched fluorescence: Tryptophan fluorescence intensity of native avidin (180 μL, final concentration 5 μM) was measured at an excitation wavelength of 270 nm and the emission was recorded at 300-400 nm. Free initiator (10 μL, final concentrations 5.12 mM) or free initiation inhibitor (10 μL, final concentrations 5.12 mM) were added to avidin solution and florescence intensities were measured. After the fluorescence intensities were recorded with free initiator or free initiation inhibitor, biotin (10 μL, final concentration 10 μM) was added to the mixture and fluorescence intensities were measured again.

Biotin and biotin PEG binding kinetics: Kinetic measurements of avidin-pCBMA conjugates with biotin and biotin-PEG substrates were taken using a stopped-flow spectrometer with fluorescence detection (Applied Photophysics SX20). The dead time of the instrument was 2 ms. The excitation wavelength was 270 nm with 5 nm bandwidth. Instrument permitted to collect 1000 data points throughput the reaction (0.1-450 s). For all experiments avidin concentration was 0.5 μM (final) and biotin or biotin-PEG concentration was 5.0 μM (final). Reactions were initiated by mixing equal volumes of avidin with its substrates in 0.1 M phosphate buffer, pH 8. Fluorescence was measured in volts. Data were fit to single exponential functions using F(t)=F_{∞}·ΔFexp(−k_{obs}t) equation, where, k_{obs} is the observed first-order rate constant, F_{∞} is the final value of fluorescence and ΔF is the amplitude. In case of native avidin kinetics the data were fit to single exponential functions using F(t)=F_{∞}+ΔFexp(−k_{obs}t) equation, where, k_{obs} is the observed first-order rate constant, F_{∞} is the final value of fluorescence and ΔF is the amplitude. All data analysis was performed in Microsoft Excel.

Biotin-Protein binding kinetics: For biotinylated protein binding kinetics avidin conjugates (0.5 μM final) were mixed with biotin-protein (5.0 μM final) in a stopped-flow accessory on PTI QuantaMaster-400 fluorometer (Horiba Instruments Inc.). The dead time of the instrument was 60 ms. The excitation wavelength was 295 nm (to selectively excite tryptophan residues) with a 20 nm bandwidth (Skelly et al., FEBS Lett 262:127-130, 1990). Excitation occurred through a 1.96-mm path in the stopped-flow optical cell, and emission was measured through a 7.68-mm path. 10 data points per second were collected throughout the reaction (15-300 s). Reactions were initiated by mixing equal volumes of avidin with its biotinylated substrates in 0.1 M phosphate buffer, pH 8. Data were fit to single exponential functions using the same equation used for biotin and biotin-PEG binding kinetics. All data analysis was performed in Microsoft Excel.

Instrumentation and Sample Analysis Preparations: ¹H and ¹³C NMR were recorded on a spectrometer (300 MHz, 75 MHz, Bruker Avance™ 300), with deuterium oxide (D₂O), DMSO-d₆, and CDCl₃. Routine FT-IR spectra were obtained with a Nicolet Magna-IR 560 spectrometer (Thermo). UV-VIS spectra were obtained and used for protein concentration determination using a UV-VIS spectrometer (Lambda 2, PerkinElmer). Melting points (mp) were measured with a Laboratory Devices Mel-Temp. Number and weight average molecular weights (Mₙ and M_{w}) and the polydispersity index (M_{w}/Mₙ) were estimated by gel permeation chromatography (GPC) on a Water 2695 Series with a data processor, equipped with three columns (Waters Ultrahydrogel Linier, 500 and 250), using Dulbecco's Phosphate Buffered Saline with 0.02 wt % sodium azide as an eluent at flow rate 1.0 mL/min, with detection by a refractive index (RI) detector. Pullulan standards (PSS-Polymer Standards Service—USA Inc., Amherst, Mass.) were used for calibration. Matrix-Assisted Laser Desorption Ionization Time-of-Flight Spectrometry (MALDI-ToF MS) was performed with a Perseptive Biosystems Voyager Elite MALDI-TOF spectrometer. Dynamic Light Scattering (DLS) data were collected on a Malvern Zetasizer nano-ZS. The concentration of the sample solution was kept at 1.0 mg/mL. The hydrodynamic diameter of samples was measured three times (5 run to each measurement, reported as number distribution) in 0.1 M sodium phosphate, pH 8. Biotin and bio-PEG binding kinetics were measured using a stopped-flow spectrometer with fluorescence detection (Applied Photophysics SX20). The excitation wavelength was set to 270 nm and emission wavelength was observed at 340 nm with 5 nm bandwidth. Instrument permitted to collect 1000 data points throughput the reaction (0.1-450 s). Bio-pro binding kinetics were obtained using a stopped-flow accessory mounted on PTI QuantaMaster-400 fluorometer (Horiba Instruments Inc.). The excitation wavelength was set to 295 nm and emission wavelength was monitored at 340 nm. Ten data points per second were collected and data were acquired using FelixGX software (Horiba Instruments Inc.). For all experiments, avidin concentration was 0.5 μM (final) and biotin, bio-PEG or bio-pro concentrations were held at 5.0 μM (final). Reactions were initiated by mixing equal volumes of avidin with its substrates in 0.1 M phosphate buffer, pH 8. Fluorescence was measured in volts.

Synthesis of NHS-Functionalized double-headed ATRP initiator (FIG. 16A): 2-bromo-2-methylpropionyl-N-oxysuccinimide ester (3): N,N′-dicyclohexylcarbodimine (10.9 g, 53 mmol) in dichloromethane (10 mL) was slowly added to the solution of 2-bromo-isobutyric acid (8.0 g, 48 mmol) and N-hydroxysuccinimide (6.1 g, 53 mmol) in dichloromethane (100 mL) at 0° C., and the mixture was stirred at room temperature overnight. Precipitated urea was filtered out and the filtrate was evaporated to remove solvent. 2-bromo-2-methylpropionyl-N-oxysuccinimine ester was isolated by recrystallization in 2-propanol; yield 11.1 g (88%), mp 72-74° C. ¹H NMR (500 MHz, CDCl₃) δ 2.08 (s, 6H, C═OC(CH₃)₂Br), and 2.86 (s, 4H, succinimide) ppm; 13C NMR (125 MHz, CDCl₃) δ 25.6, 30.7, 51.2, 164.5, 168.6 ppm; IR (KBr pellete) 3499, 3004, 2985, 2942, 1811, 1780, 1738, 1425, 1371, 1252, 1210, 1125 and 1082 cm⁻¹.

Bis(2-(2-bromo-2-methylpropanamido)ethylamine 2-bromo-2-methylpropionyl-N-succinimide ester (5.3 g, 2.0 mmol) was slowly added to a solution of diethylenetriamine (1.0 g, 9.7 mmol) and triethylamine (1.4 mL, 1.0 mmol) in acetonitrile (50 mL) at 0° C., and mixture was stirred at room temperature overnight. Precipitated N-hydroxysuccinimide was filtered out and the filtrate was evaporated to remove solvent. Ethyl acetate (50 mL) was added to the mixture and the organic phase was washed with 50 wt % sodium carbonate aq. (20 mL×3) and saturated NaCl aq. (20 mL×3). The organic phase was dried with Na₂CO₃ and evaporated to remove solvent. (4) was isolated by column chromatography (silica and acetonitrile). oil compound; yield 2.7 g (69%), ¹H NMR (500 MHz, CDCl₃) δ 1.46 (broad s, 1H, amine), 1.96 (s, 12H, NHC═OC(CH₃)₂Br), 2.82 (t, 4H, J=6.0 Hz, C═ONHCH₂CH₂NH), 3.36 (td, J=6.0, 5.5 Hz, C═ONHCH₂CH₂NH), 7.15 (broad s, 2H, amide) ppm; ¹³C NMR (125 MHz, CDCl₃) δ 32.5, 40.1, 48.2, 62.9, 172.2 ppm; IR (NaCl plate) 3506, 3340, 3066, 2975, 2930, 2852, 1647, 1533, 1463, 1387, 1368, 1288, 1193, 1110 and 1050 cm⁻¹.

4-(bis(2-(2-bromo-2-methylpropanamido)ethyl)amino)-4-oxobutanoic acid (5): Succinic anhydride (600 mg, 6.0 mmol) and triethylamine (840 μL, 6.0 mmol) was added to the solution of 1 (2.2 g, 5.5 mmol) in acetonitrile (50 mL), and mixture was stirred at room temperature overnight. After solvent was evaporated, ethyl acetate (50 mL) was added to the mixture. The organic phase was washed with 1 N HCl aq. (20 mL×3) and saturated NaCl aq. (20 mL×3). The organic phase was dried with MgSO₄ and evaporated to remove solvent. (5) was isolated by column chromatography (silica and acetonitrile:chloroform (1/3 volume ratio)le). Oil compound; yield 2.2 g (80%), ¹H NMR (500 MHz, CDCl₃), δ 1.92 and 1.95 (s, 12H, to NHC═OC(CH₃)₂Br), 2.67-2.74 (m, 4H, NC═OCH₂CH₂COOH), 3.44-3.50 (m, 4H, C═ONHCH₂CH₂NC═O), 3.54 and 3.58 (t, 4H, J=6.0 Hz, C═ONHCH₂CH₂NC═O), 7.27 and 7.36 (broad t, J=5.5 Hz, NHC═OC(CH₃)₂Br) ppm; ¹³C NMR (125 MHz, CDCl₃) δ 28.4, 29.4, 32.0, 32.2, 39.0, 39.4, 45.5, 47.4, 60.9, 61.5, 172.9, 173.0, 173.5, 176.1 ppm; JR (NaCl plate) 3347, 2981, 2932, 1782, 1725, 1647, 1532, 1459, 1428, 1368, 1223 and 1194 cm⁻¹.

4-(bis(2-(2-bromo-2-methylpropanamido)ethyl)amino)-4-oxobutyloyl-N-oxysuccinimide ester (double-headed ATRP initiator): To a solution of (5) (2.0 g, 2.0 mmol) in acetonitrile (50 mL), di(N-succinimidyl) carbonate (1.1 g, 4.3 mmol) and triethylamine (560 μL, 4.0 mmol) were added and the mixture was stirred at room temperature overnight. After the solvent evaporated, double-headed ATRP initiator was isolated by column chromatography (silica, acetone:chloroform (1/4 volume ratio)). Oil compound, yield 2.2 g (77%), ¹H NMR (500 MHz, CDCl₃), δ 1.92 and 1.94 (s, 12H, NHC═OC(CH₃)₂Br), 2.82-2.85 (m, 6 I-1, succinimide and NC═OCH₂CH₂COOSuc), 2.97 (t, 2H, J=5.8 Hz, NC═OCH₂CH₂COOSuc), 3.43-3.489 (m, 4H, C═ONHCH₂CH₂NC═O), 3.53 and 3.58 (t, 4H, J=6.1 Hz, C═ONHCH₂CH₂NC═O), 7.19 and 7.30 (broad t, J=6.0 Hz, NHC═OC(CH₃)₂Br) ppm; ¹³C NMR (125 MHz, CDCl₃), 525.6, 26.8, 27.8, 32.0, 32.2, 39.1, 39.3, 45.7, 47.5, 60.8, 61.7, 168.2, 169.1, 171.7, 172.8, 172.9 ppm; IR (NaCl plate) 3379, 2932, 2870, 1815, 1784, 1739, 1647, 1531, 1460, 1429, 1370, 1288, 1209, 1112, 1108 and 1080 cm⁻¹.

Synthesis of free single pCBMA polymers: Free single initiator (N-2-bromo-2-methylpropionylW-alanine) was synthesized as described elsewhere (Murata et al. 2013, supra). 1.2 mg of free single initiator (0.005 mmol) and CBMA (116 mg, 0.5 mmol for pCBMA100) were dissolved in sodium phosphate buffer (9 mL, 0.1 M, pH 8). Initiator/monomer solution was sealed with rubber septum and bubbled with nitrogen for 30 minutes. 60 μL of sodium ascorbate (20 mg/mL, 0.1 mmol) and 20 μL of HMTETA were added to deoxygenated 1.2 mL of CuCl₂ (50 mM) and bubbled for 5 minutes. 1 mL of deoxygenated copper catalyst was added to a solution of deoxygenated initiator/CBMA and allowed to react for 1 hour at room temperature. The reaction was stopped upon exposure to air free polymers were purified through dialysis (3.5 kDa MWCO) against deionized water for 24 hours at 4° C. and then lyophilized.

Synthesis of free double pCBMA polymers: 2.5 mg of free dual initiator (5) (0.005 mmol) and CBMA (242 mg, 1 mmol for pCBMA₁₀₀) were dissolved in sodium phosphate buffer (9 mL, 0.1 M, pH 8). The solution of initiator/monomer was sealed with rubber septum and bubbled with nitrogen for 30 minutes. In α-separate flask, 1.2 mL of CuCl₂ (50 mM) was bubbled under argon for 2 minutes. 60 μL of sodium ascorbate (20 mg/mL, 0.1 mmol) and 20 μL of HMTETA were added to deoxygenated 1.2 mL of CuCl₂, (50 mM) and bubbled for another 5 minutes. 1 mL of deoxygenated copper catalyst was added to a initiator/monomer solution and allowed to react for 1 hour at room temperature. The reaction was stopped upon exposure to air. Free polymers were purified through dialysis (3.5 kDa MWCO) against deionized water for 24 hours at 4° C., and then lyophilized.

Acid hydrolysis of free single and deal pCBMA polymers: 20 mg of both single and double-headed initiators “grown from” pCBMA polymers were placed in hydrolysis tubes and dissolved in 6 N HCl (6 mL). After five freeze-pump-thaw cycles, the samples were kept at 110° C. under vacuum for 24 hours. The cleaved polymers were dialyzed against deionized water at room temperature, using 1 kDa molecular weight cut off dialysis tubing and then lyophilized. The molecular weight and dispersity of polymers were measured by gel permeation chromatography (GPC).

### Example 2—Avidin Conjugate Synthesis and Characterization

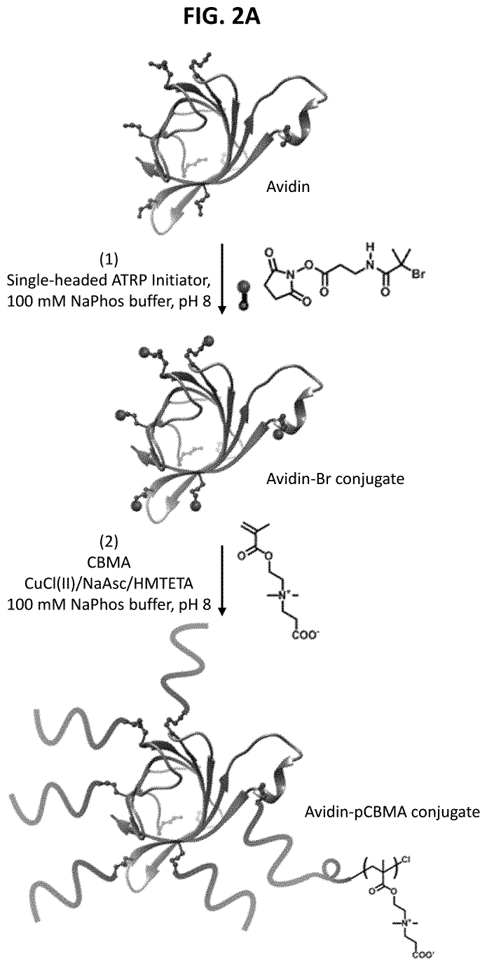

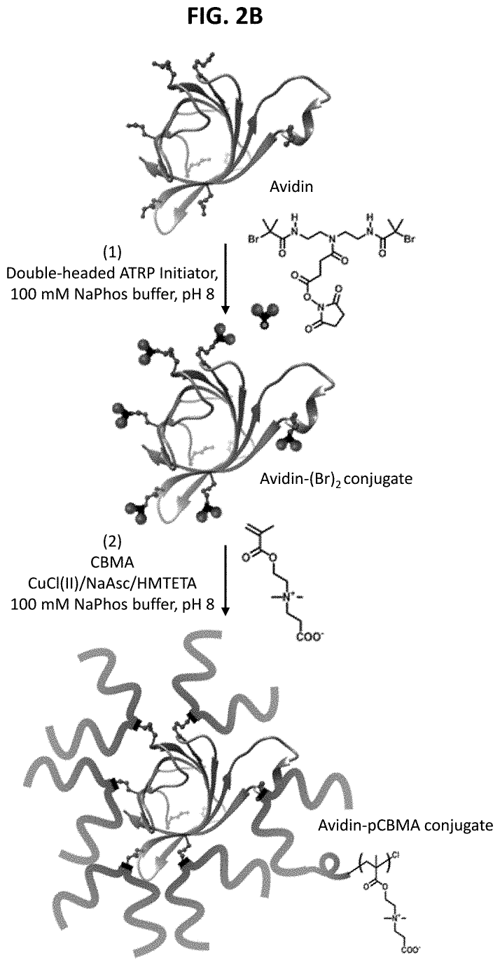

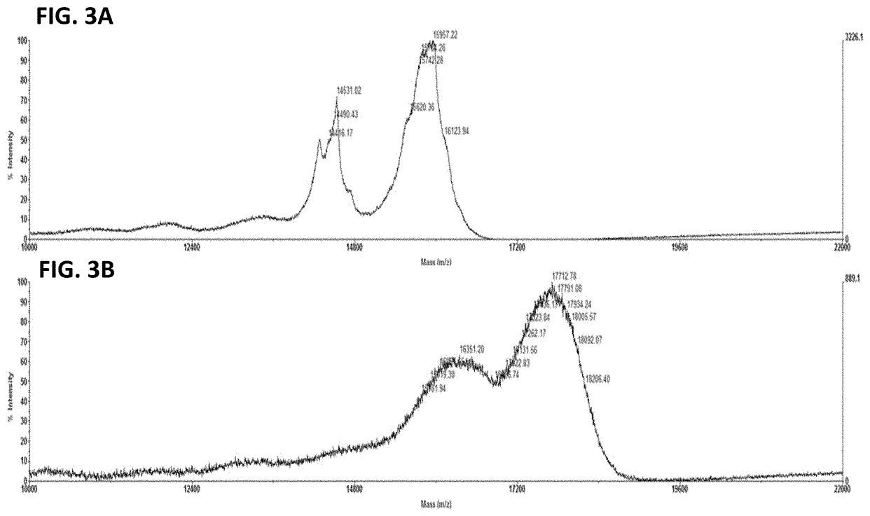

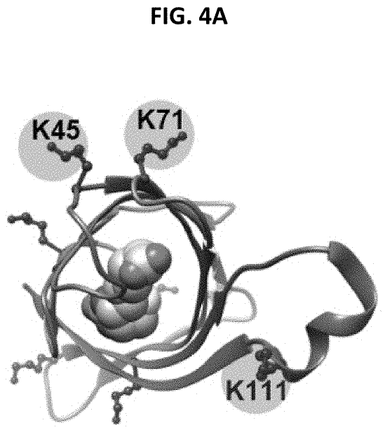

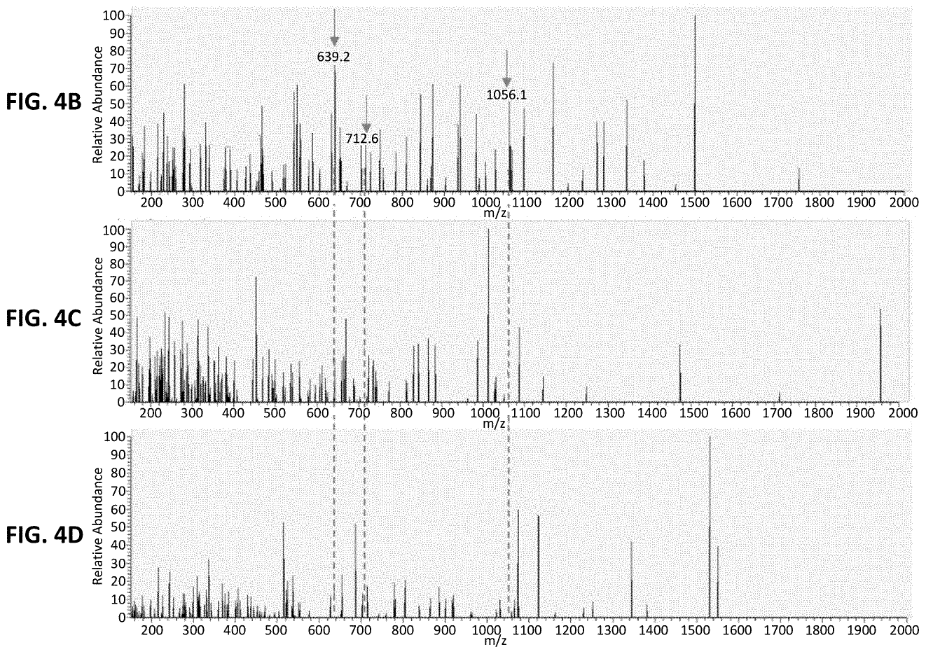

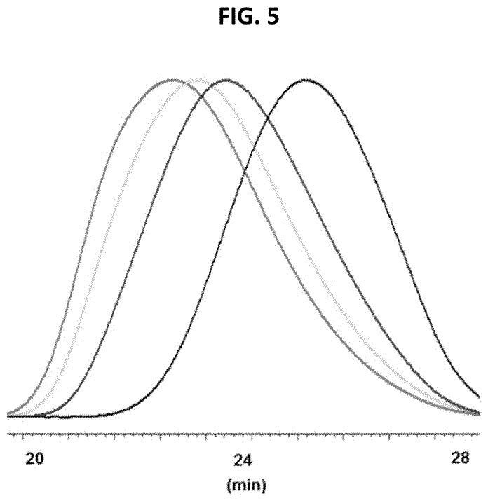

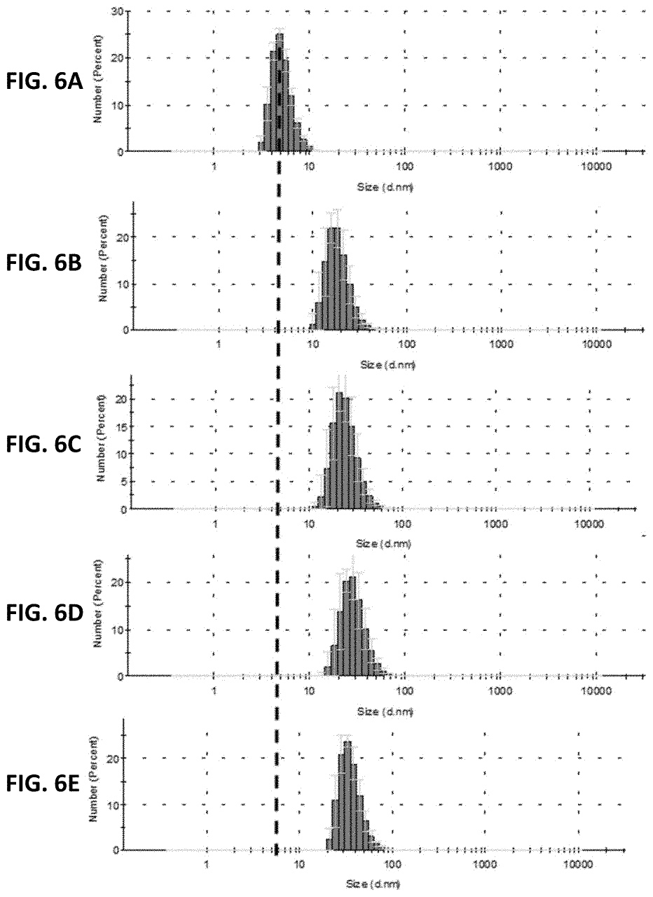

To study the impact of polymer chain length on the permeation of molecules through polymer layers on the surface of bioconjugates, avidin-polymer conjugates were synthesized by growing pCBMA directly from the surface of from avidin. Avidin is a tetrameric protein, with each monomer containing 10 primary amine groups, (1 α-amine group at the N-terminus and 9 ε-amine groups on lysine residues). The hydrophilic and zwitterionic polymer, pCBMA, has non-fouling properties and thus repels proteins both in vitro and in vivo (Lin et al., supra). pCBMA has been attached to several proteins without compromising functionality (Keefe and Jiang, Nature Chem 4:59-63, 2012). Native avidin was modified with an amine-reactive N-2-bromo-2-methylpropanoyl-β-alanine N′-oxysuccinimide bromide ATRP initiator from which a single polymer chains of pCBMA were grown (FIG. 2A). Matrix-assisted laser desorption/ionization time of flight mass spectrometry (MALDI-ToF-MS) showed that an average of 8 initiators were attached per avidin monomer (FIG. 3). The three-dimensional structure of avidin (PDB:2avi) shows that lysines 45, 71, and 111 are located near the biotin binding pocket of avidin, and therefore are ideal modification targets for determining the rate at which ligands can penetrate attached polymers and bind to the surface of the protein (FIG. 4A). Trypsin digestion studies with peptide mapping using electrospray ionization mass spectrometry (ESI-MS; Carmali et al. 2017, supra) on the avidin-initiator complexes confirmed that the targeted amino acids (K45, K71 and K111) were covalently modified with ATRP initiator (FIG. 4B and TABLE 1). After confirming K45, K71, and K111 modification, the polymerization conditions were varied by changing the monomer concentration to synthesize avidin-pCBMA conjugates with four different target lengths or degrees of polymerization (DP): 50, 100, 150 and 200) (FIG. 2A). To measure the dispersity and molecular weights of the polymers, avidin conjugates were acid hydrolyzed to cleave the polymers from the protein surface, followed by molecular weight analysis using gel permeation chromatography (TABLE 2 and FIG. 5). Molecular weights of the conjugates were estimated using a bicinchonic acid (BCA) assay (Murata et al. 2013, supra). The hydrodynamic diameters of (DO of avidin conjugates were determined using dynamic light scattering (DLS). As expected, the molecular weights and Dh of the conjugates increased with increasing length of grafted pCBMA (TABLE 2 and FIG. 6). Spectrophotometric assay based on the binding of 4′-hydroxyazobenzene-2-carboxylic acid (HABA) was used to determine the binding activity of avidin conjugates (TABLE 3) (Green, Biochem J 94:21-24, 1965). The functionality of each conjugate could then be assessed.

### Example 3—Tryptophan Fluorescence Changes of Avidin Upon Binding Biotin

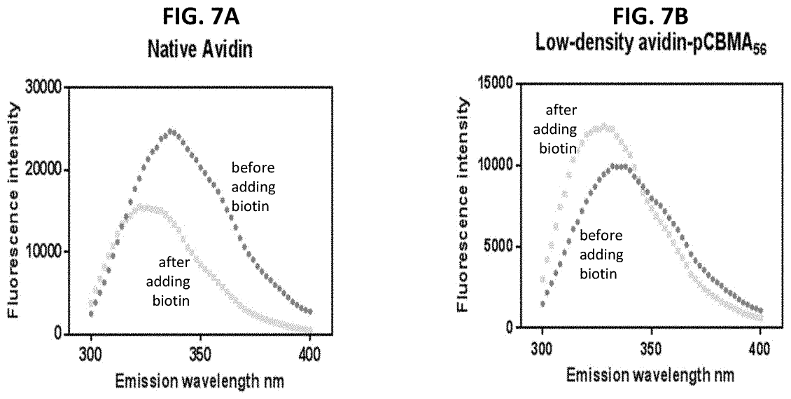

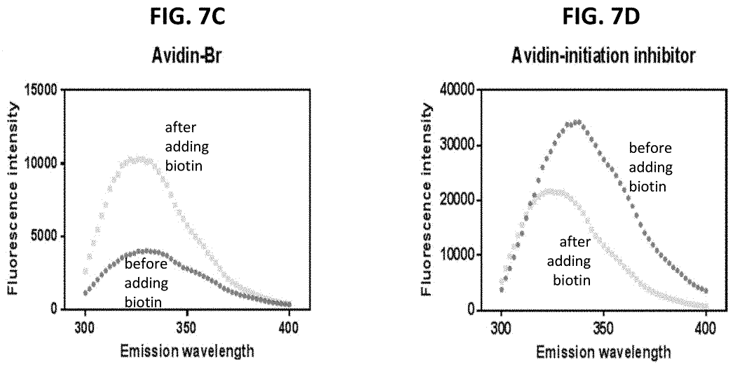

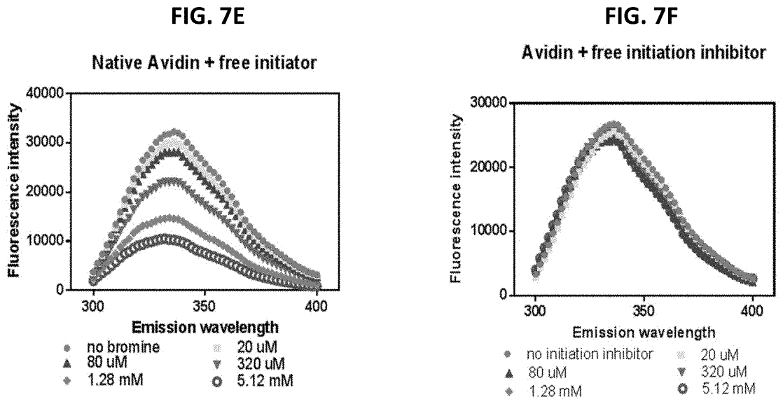

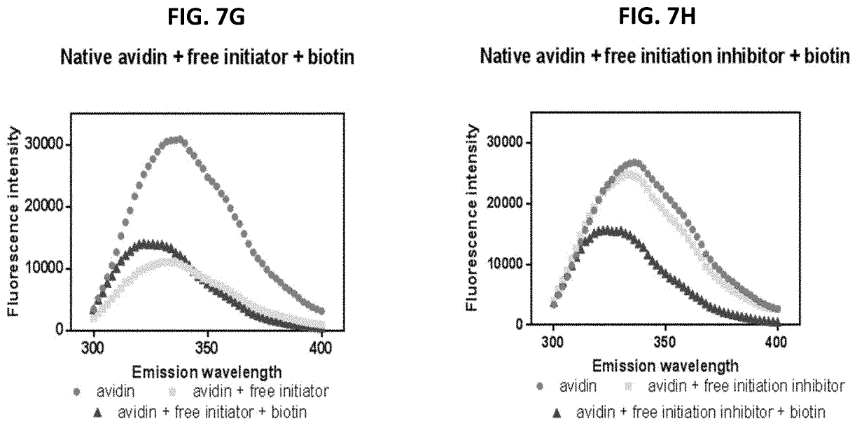

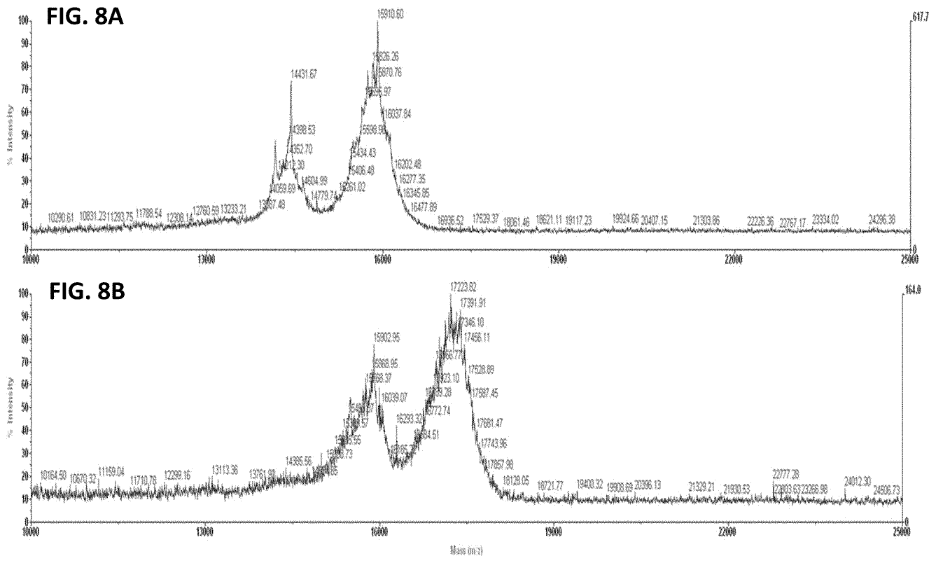

Work described elsewhere has shown that, upon biotin binding, the intrinsic tryptophan fluorescence of avidin is decreased from 337 to 324 nm with a blue shift in emission (Kurzban et al., Biochem 28:8537-8542, 1989; and Kurzban et al., J Protein Chem 9:673-682, 1990). Surprisingly, when biotin was added to a solution of any avidin-pCBMA conjugate, an increase in tryptophan fluorescence was observed (FIG. 7B). A series of key experiments (see, FIGS. 7A, 7B, and 8) demonstrated that the increase in intrinsic fluorescence of avidin-pCBMA conjugates after binding biotin was not caused by structural changes upon protein modification, but was a result of quenching of tryptophan fluorescence by the halide-terminated initiator and polymers (Kurzban et al. 1989, supra). It was speculated that the fluorescence quenching of tryptophan residues by the bromide group is possibly driven by contact quenching. A tryptophan electron at the excited singlet state is caused to crossover to triplet state by bromide group, and as soon as it crosses to the triplet state it is immediately quenched by either the bromide group or oxygen (Sarker et al., J Phys Chem A 107:6533-6537, 2003; and Grewer and Brauer, J Phys Chem 98:4230-4235, 1994). It was suggested that while the bromide group on the ATRP initiator acts as a quencher and causes decreased initial fluorescence, biotin acts as a dequencher and leads to an increase of fluorescence upon binding. This discovery provided a handle through which, using complex stopped flow fluorescence analysis, the rate at which biotin binds to avidin-pCBMA complexes could be tracked.

### Example 4—Ligand Binding Rate to Avidin Through Polymer Sieves

In general, kinetic studies of protein adsorption to polymer-modified surfaces have used surface plasmon resonance (SPR) or quartz crystal microbalance (QCM) (Green et al., Biomaterials 18:405-413, 1997; and Hook et al., Analytical Chem 73:5796-5804, 2001; and Green et al., Biomaterials 21:1823-1835, 2000). While very sensitive, factors such as polymer density, thickness, viscosity, protein size, and difficulties in detection of low molecular weight substrates can limit these techniques (Luan et al., Analytical Chem 89:4184-4191, 2017; and Ahmed et al., Cancer Genomics Proteomics 7:303-309, 2010).

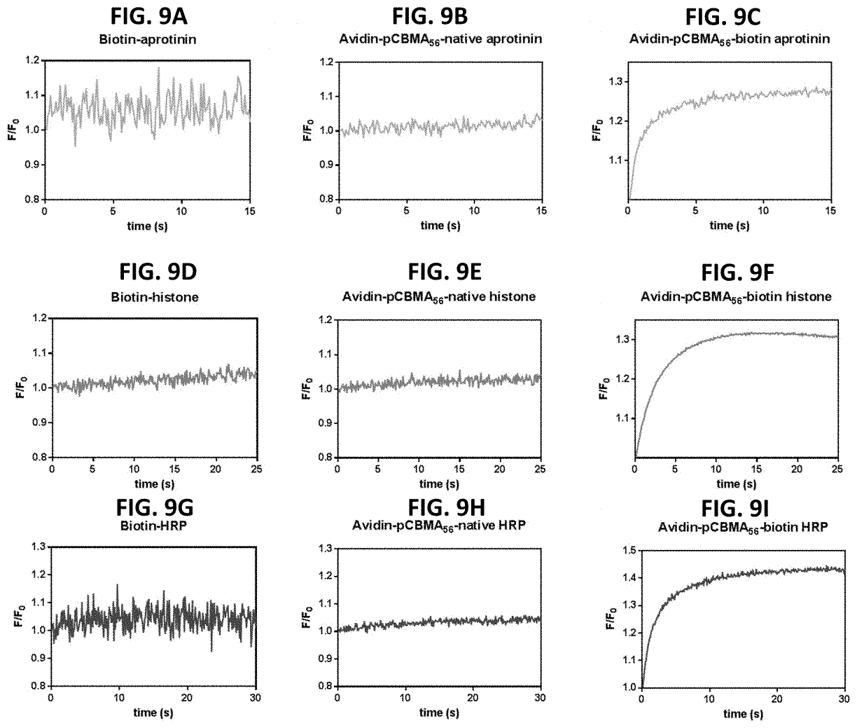

As described herein, stopped-flow kinetic techniques were used to develop an assay to determine the rate of binding of biotinylated substrates to avidin and avidin-pCBMA conjugates. The rate of binding was measured under first-order reaction conditions in which the concentration of biotin was in molar excess. All of the avidin-pCBMA conjugates bound biotin much more slowly (0.63-0.73%) than native avidin (TABLE 4). It is worth noting that this drop in biotin binding kinetics was not due to avidin conjugates losing their binding activity, but was driven by a sieving effect created by covalently attached polymers on the avidin surface (TABLE 3). A surprisingly small difference in the rate of biotin binding to the conjugates as a function of polymer chain length also was observed (lobs varied from 0.763-0.668 s⁻¹). Thus, studies were conducted to explore how diffusion of the molecules toward the protein surface changed as a function of the size of the diffusing molecules. A series of proteins of varying sizes were biotinylated: aprotinin (M_{w} 6.5 kDa), histone (M_{w} 21.5 kDa), and horse radish peroxidase (HRP, M_{w} 44.2 kDa) (TABLE 5). In addition to size, a criteria in selecting these protein substrates was to keep the number of tryptophan residues to a minimum so the assays for biotin binding could be performed. Since tryptophan residues are extremely sensitive to their environment (Ghisaidoobe and Chung, Int J Mol Sci 15:22518-22538, 2014; and Vivian and Callis, Biophysical J 80:2093-2109, 2001), when bound to avidin the environment of tryptophan residues on protein substrates will also change leading to changes in emission spectrum. Two of the selected proteins (aprotinin and histone) do not have any tryptophan residues, and HRP has only two tryptophan residues that do not interfere with the binding emission spectrum (FIG. 9). The data revealed that the permeation rate of these proteins was sharply dependent on the size of the diffusing protein. The smallest protein substrate (aprotinin) had the fastest binding rate. A similar trend was observed in a another study of protein permeation through hydrogel membranes, where the diffusion coefficient of the proteins through a certain mesh sized hydrogels was highly dependent on the protein size (Burczak et al., Biomaterials 15:231-238, 1994). Interestingly, binding rates of the biotinylated proteins were again barely dependent on the molecular weight of the polymer that had been grown from the surface of avidin. (TABLE 4). Similar results were observed in a study of protein adsorption as a function of PEG grafting density, molecular type (linear and star-like), molecular weight and adsorbing protein size (Sofia et al., Macromolecules 31:5059-5070, 1998). That work demonstrated that covering silicon surfaces with at least half-overlapping PEG chains is important for protein repulsion, and that the overlap is independent of PEG molecular weight. However, to achieve higher overlap and protein repulsion for all PEG molecular weights, higher grafting density was needed. In addition, the amount of a protein adsorbed at a given grafting density and PEG molecular weight correlated with the size of the protein. Studies were therefore carried out to determine whether the shape of the ligand and the grafting density of pCBMA on avidin complexes impacted the rate of binding.

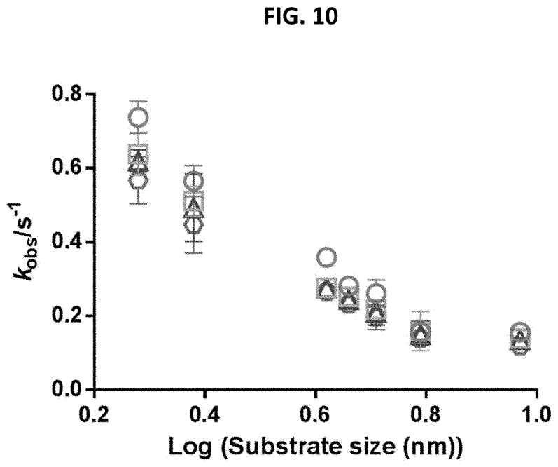

Four PEG polymers of different sizes were selected (biotin-PEG: 550 Da, 5 kDa, 10 kDa, and 30 kDa) (TABLE 5). The binding rate of the biotin-PEG 550 Da to native avidin was 80% slower than biotin itself. The biotin-PEG 550 Da bound to avidin conjugates 3-15% slower than biotin (TABLE 4). A ten-fold increase in the size of the PEG-biotin chain (biotin-PEG 5 kDa) decreased the rate of binding to avidin by approximately half relative to the rate of binding of biotin, while diffusion of biotin-PEG 10 kDa was 78-79% slower than biotin. Lastly, the binding rate of biotin-PEG 30 kDa to avidin was 80-82% slower than that of biotin. In all cases, it was observed that larger biotin-PEG substrates bound more slowly to the active site. Again, a pronounced dependence of permeation rate of linear PEGs on the pCBMA molecular weight that was attached to avidin was not observed (TABLE 4). Hydrodynamic diameters of both biotin-protein and biotin-PEG substrates were measured using DLS, showing that the smallest substrate was PEG 550 Da, followed by aprotinin, PEG 5K, histone, HRP, PEG 10K and finally PEG 30K (TABLES 5 and 6). After analyzing both the Dh and permeation rates, it was observed that the permeation rate for biotinylated aprotinin (Dh 2.4 nm) was slower than the permeation rate of biotin-PEG 550 Da (Dh 1.9 nm) (FIG. 10). These observations led to the hypothesis that for single-headed initiator “grown from” avidin conjugates, it was the ligand size, not shape, that was important in determining the permeation rate through polymer shell. These data suggested that increasing polymer density around the active site of avidin may drive more effective sieving. Unfortunately, varying polymer density in protein-ATRP from single sites was not possible until the work described herein was carried out.

### Example 5—Synthesis and Characterization of a High-Density Conjugates

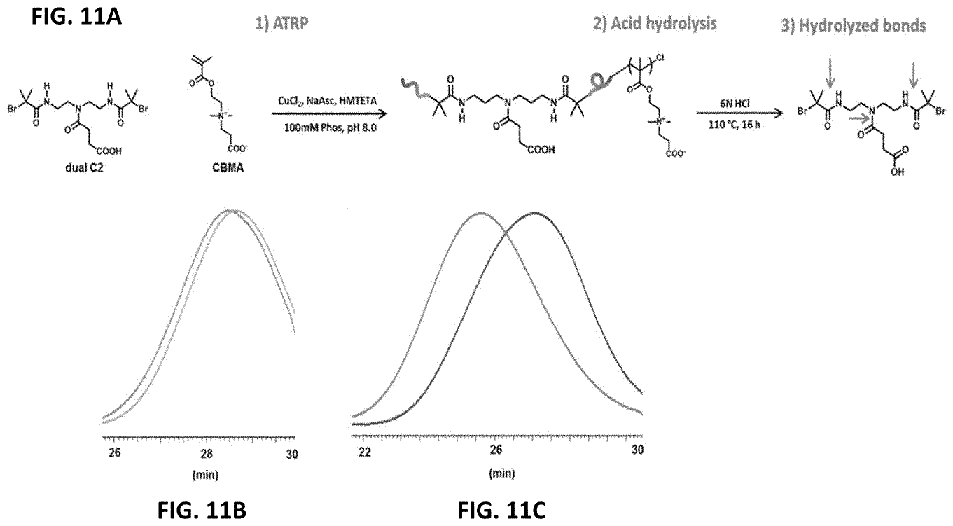

To increase polymer density, a novel double-headed ATRP initiator was synthesized that allowed the growth of two polymer chains from each initiation site (Example 1). First, polymerization was performed from unattached double-headed ATRP initiator to confirm the growth of both polymer chains from one initiator and to optimize the conditions for conjugate synthesis (FIG. 11). GPC before and after acid hydrolysis of the synthesized polymers was used to show that one double-headed initiator led to the growth of two polymer chains in solution (TABLE 7).

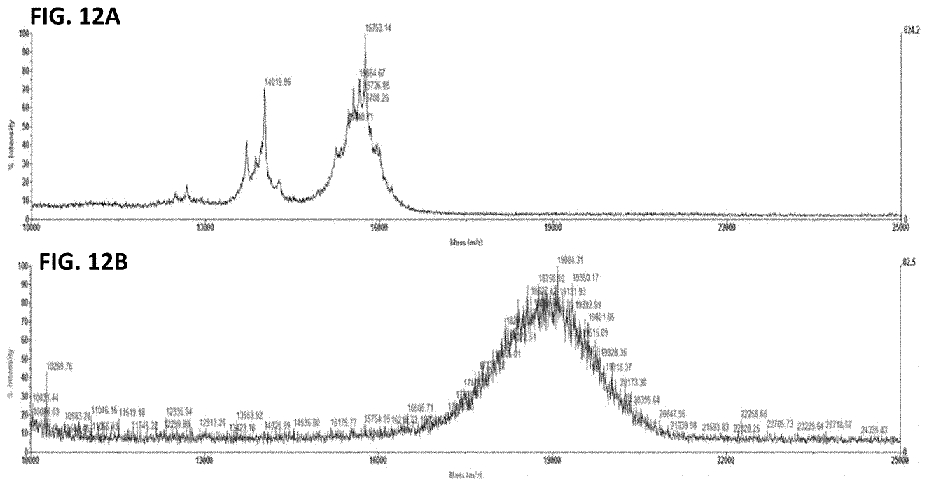

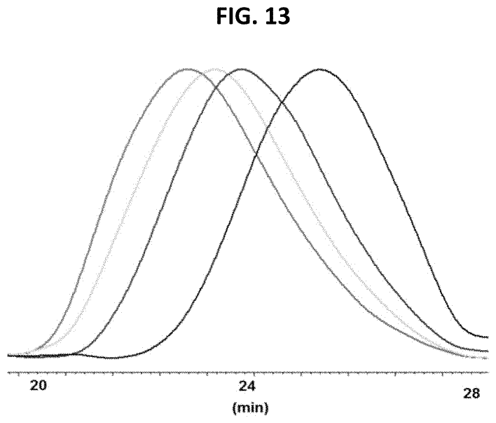

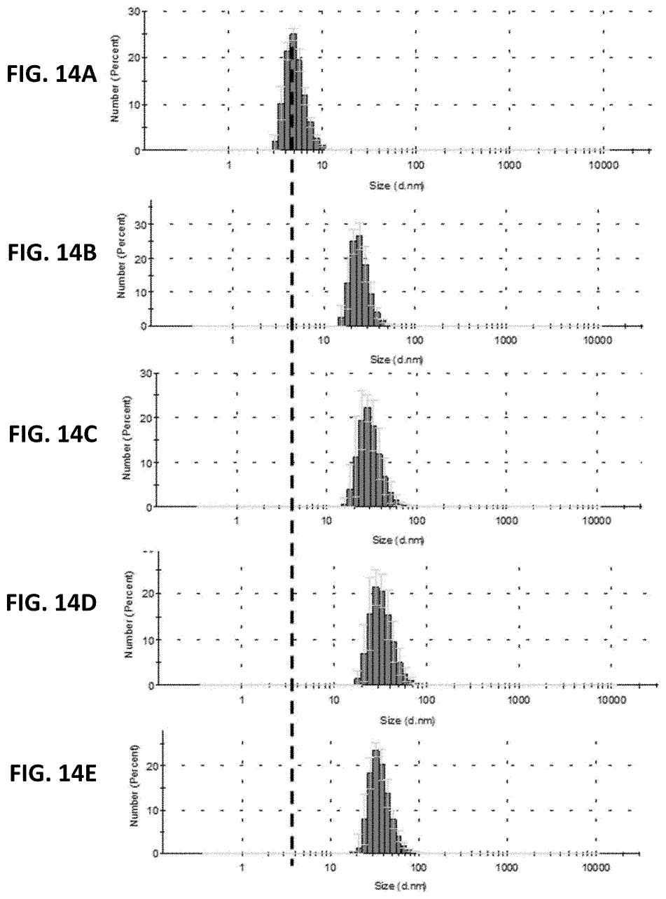

Next, the double-headed ATRP initiator was reacted with primary amines on the surface of avidin (FIG. 2B), and MALDI-ToF was used to show that there were an average of 7 double-headed initiators on each avidin monomer (FIG. 12). Unsurprisingly, the larger initiator lost its ability to react with at least one lysine previously accessible by the single-headed initiator. The initiation sites that the double-headed initiator targeted were well-distributed on the avidin surface and a polymer density of one polymer per 0.5 chains/nm² for the double-headed initiator “grown from” conjugates was calculated. This led to a 1.8-fold higher pCBMA grafting density as compared to the single-headed initiator derived conjugates (0.29 polymer chains/nm²). Trypsin digestion followed by ESI-MS was used to determine that the K45, K71, and K111 residues near the binding site had reacted with double-headed ATRP initiator (FIG. 3D and TABLE 1). The molecular weight and degree of polymerization of the high-density avidin-pCBMA conjugate was then characterized. As expected, the molecular weights of the high-density conjugates were larger than the molecular weights and hydrodynamic sizes of the low-density conjugates for the same degree of polymerization. Next, GPC was used for determination of cleaved polymer molecular weight and dispersity (TABLE 8 and FIG. 13). DLS was used for hydrodynamic diameter measurements (TABLE 8 and FIG. 14). A spectrophotometric method was used to determine HABA binding activity of high-density avidin conjugates (TABLE 3).

### Example 6—Polymer Grafting Density Effect on Binding Kinetics

High-density avidin-pCBMA conjugates had a two-fold decrease in biotin binding rate compared to low-density conjugates (TABLE 9) (lobs differed from 0.345-0.299 s⁻¹ versus 0.763-0.668 s⁻¹, respectively). Doubling the grafting density from each initiation site on avidin had a marked effect on the binding rate of biotinylated macromolecules. As expected, aprotinin had the fastest permeation rate, followed by histone and then HRP. The permeation rate of the biotinylated proteins through the high-density polymer shell to the avidin binding site was about ten-fold lower than the permeation rate for the low-density avidin-pCBMA conjugates. Surprisingly, the high-density avidin-pCBMA conjugates still bound the largest protein substrate (HRP), although at a decreased rate. This was consistent with a previous theoretical study, which postulated that just covering the surface with PEG polymers was not sufficient to prevent the proteins from reaching the surface (Szleifer, Biophys J 72:595-612, 1997). That study revealed that proteins can permeate through polymer layers and localize between polymer chains. The studies described herein revealed a strong dependence of biotin-protein diffusion on the pCBMA grafting density. The rates of binding for the high-density avidin-pCBMA conjugates were also not strongly impacted by the molecular weight of the grafted pCBMA (TABLE 9). Similar results were observed in a theoretical characterization of protein resistance properties of PEG chains attached to hydrophobic surfaces, by calculating the steric repulsion free energy initiated by protein compressing PEG chains and hydrophobic interaction free energies as a function of polymer grafting density and molecular weight (Jeon et al., J Colloid Interface Sci 142:149-158, 1991). Higher grafting density exhibited stronger protein repulsion due to the compression of PEG chains, and thus was more important than polymer molecular weight in preventing protein adsorption.

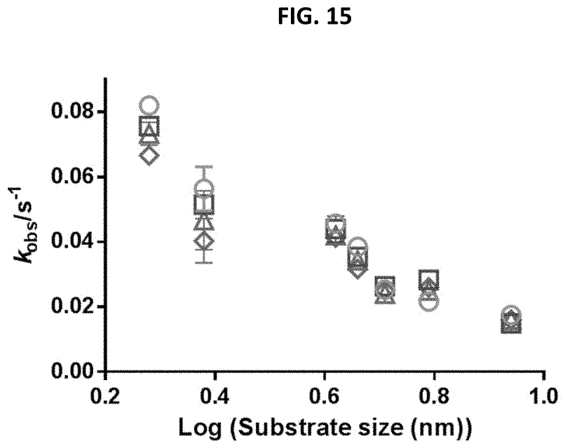

The binding kinetics for the interaction between high-density avidin-pCBMA conjugates and biotinylated-PEG substrates was sharply dependent on the size of the PEG (TABLE 9 and FIG. 15). The binding rates of all molecules to these conjugates were ten-fold slower than for the low-density conjugates. Again, it was found that molecule shape was unimportant in diffusion through grafted polymers, and diffusion rate changed as a function of ligand size.

As described herein, the rates of binding of globular and linear macromolecules to a protein surface through a layer of covalently attached polymers have been quantified, for the first time. Surface initiated ATRP was used to synthesize avidin-pCBMA bioconjugates that were employed to investigate the role of polymer molecular weight and grafting density in shielding protein surface from molecule penetration. Stopped-flow kinetics proved to be a powerful tool in measuring the binding rates of biotinylated molecules of varying shape and size to the avidin binding pocket through grafted pCBMA polymers. A unique double-headed ATRP initiator was generated, enabling the synthesis of protein-polymer bioconjugates with high grafting densities, without the need to change the protein itself. This chemistry may provide new avenues in creation of bioconjugates covered with dense polymer shells using double, triple or even multi-headed initiators. It was concluded that there appears to be no specific pCBMA molecular weight that is necessary to affect the ligand binding rate, at least in the range of substrate sizes and pCBMA lengths studied. Instead, for a given pCBMA molecular weight, the grafting density slowed the diffusion and binding of ligands to the protein active site. Additionally, it was discovered that molecule permeation depends on substrate size, independent of substrate shape.

### Example 7—Intrinsic Tryptophan Fluorescence Changes of Avidin Upon Biotin Binding

To demonstrate how the permeation rate of biotinylated molecules toward the avidin surface will vary as a function of polymer chain length and density grafted from the surface of avidin, studies were conducted to observe changes in intrinsic tryptophan fluorescence upon biotin binding. Upon biotin binding, intrinsic tryptophan fluorescence of avidin was decreased from 337 to 324 nm and a blue shift in emission was observed (Kurzban 1989, supra). However, fluorescence changes of avidin-pCBMA conjugates were unexpected. When biotin was added to a solution of avidin-pCBMA conjugate, an increase in tryptophan fluorescence was observed (FIG. 7B). In order to determine whether these changes in fluorescence were occurring due to ATRP initiator attachment or ATRP from the avidin surface, the fluorescence changes of ATRP initiator-modified avidin when it binds biotin were tested. An increase in the intrinsic fluorescence of initiator-modified avidin conjugate upon biotin addition was observed (FIG. 7C). Next, studies were conducted to investigate whether the fluorescence changes were due to structural changes of the protein after initiator attachment, or were caused by ATRP initiator. Avidin was modified with an initiation inhibitor. Structurally ATRP initiator-like but with no halide atom, this compound reacts with amine groups on avidin without initiating and leading to polymer growth (Carmali et al., supra). After immobilization, it was determined using MALDI-ToF that there were an average of 8 initiation inhibitor molecules per avidin monomer (FIG. 8). When the changes in intrinsic fluorescence upon biotin addition were tested, it was observed that in the case of initiation inhibitor modified avidin, the fluorescence of tryptophan residues decreased in a manner similar to native biotin-bound avidin (FIG. 7D). These results suggested that the increase in intrinsic fluorescence of avidin conjugates after binding biotin, was not caused by structural changes upon protein modification.

It was hypothesized that while a bromide group on ATRP initiator acts as a quencher and causes decreased initial fluorescence, biotin acts as a dequencher and leads to an increase of fluorescence upon binding. To test this, studies were conducted to determine tested whether free ATRP initiator could quench tryptophan fluorescence. Free ATRP initiator was used for this assay because unlike ATRP initiator, it lacks N-hydroxy succinimide ester and thus will not react with protein. Increasing concentrations of free initiator were added to native avidin solution and intrinsic fluorescence was measured. With increasing concentration of free initiator, a higher degree of fluorescence quenching was observed (FIG. 7E). Further studies investigated whether N-2-methylpropanoyl-O-alanine (free initiation inhibitor) will quench tryptophan fluorescence in a similar manner to free initiator. None of the tested concentrations of initiation inhibitor had an effect on fluorescence of native avidin, suggesting that alkyl-bromide is able to quench tryptophan fluorescence (FIG. 7F).

Further experiments were carried out to determine whether biotin is capable of dequenching the fluorescence, and whether the increase in fluorescence is caused by biotin binding to avidin conjugates. Native avidin tryptophan fluorescence was quenched with 5.12 mM free initiator, followed by biotin addition (FIG. 7G). The results of these studies showed that biotin restored the quenched tryptophan fluorescence, as an increase in fluorescence was observed as biotin bound to native avidin. The experiment was repeated with native avidin and free initiation inhibitor, demonstrating the changes in fluorescence when biotin was added. As shown in FIG. 7H, free initiation inhibitor did not have effect on fluorescence, while biotin binding decreased the tryptophan fluorescence.

### Example 8—Bio-Pro Binding to Avidin Conjugates

To avoid unexpected changes in the emission spectrum, protein binding kinetics were measured at an excitation wavelength of 295 nm and emission of 340 nm to selectively excite only tryptophan residues (Skelly et al., supra). Both aprotinin and histone do not have tryptophan residues, while HRP has two tryptophan residues. As shown in FIGS. 9A, 9D, and 9G, the emission spectra of these proteins did not change over time. Next, studies were conducted to explore whether mixing native aprotinin, histone, or HRP with an avidin conjugate will lead to changes in the emission spectrum using a fluorimeter with stopped-flow accessory. Upon mixing equal volumes (200 μL) of avidin-pCBMA₅₆ and native aprotinin, histone, or HRP, the fluorescence emission spectra of tryptophan residues on avidin did not change (FIGS. 9B, 9E, and 9H). These data suggested that native proteins do not bind to avidin conjugates and do not cause any changes in emission spectrum. The changes in the spectra were further tested when avidin conjugate was mixed with biotinylated proteins (FIGS. 9C, 9F, and 9I), demonstrating that the emission spectrum of the avidin conjugate increased immediately, suggesting binding.

TABLE 1
Peak analysis of peptide fragments after
trypsin digestion for native avidin.

| | Peptide | Expected | Observed |
|---|---|---|---|
| Protein | Fragment | Mass (m/z) | mass (m/z) |
| K45 | ¹GEFTGTYTTAVT | 1058.1 | 1056.1 |
| | ATSHEIK | [M + 3ACN + 2H]²⁺ | |
| | (SEQ ID NO: 1) | | |
| K71 | ¹TQPTFGFTVNWK | 713.8 | 712.6 |
| | (SEQ ID NO: 2) | [M + 2H]²⁺ | |
| K111 | ¹SSVNDIGDDWK | 639.2 | 639.2 |
| | (SEQ ID NO: 3) | [M + ACN + 2H]²⁺ | |

TABLE 2
Characterization of low-density avidin-pCBMA conjugates

| | | | Estimated | Cleaved | Estimated |
|---|---|---|---|---|---|
| | Polymerization | | conjugate | polymer^{d} | conjugate |
| | condition^{a} | | M_{w}^{c} (kDa) | Mₙ(kDa); | M_{w}^{e} (kDa) |
| Sample | [I]₀/[M]₀ | Dₕ^{b} | (BCA) | (M_{w}/Mₙ) | (GPC) |
| Avidin-pCBMA₅₆ | 1:100 | 18.7 ± 1.9 | 72 | 12.8 (1.8) | 200.0 |
| Avidin-pCBMA₁₂₁ | 1:150 | 24.8 ± 2.8 | 150 | 27.8 (1.8) | 416.3 |
| Avidin-pCBMA₁₇₀ | 1:200 | 29.2 ± 3.4 | 184 | 39.0 (1.9) | 608.8 |
| Avidin-pCBMA₂₃₂ | 1:250 | 35.7 ± 2.6 | 220 | 53.3 (1.7) | 740.9 |

^{a}Eight initiators per avidin monomer, [I]₀/[Cu(II)Cl]₀/[NaAcs]₀[HMTETA]₀ = 1:10:12:10.
^{b}Hydrodynamic diameters (number distribution) of the avidin-pCBMA conjugates was measured using dynamic light scattering with sample concentration 1.0 mg/mL in 100 mM sodium phosphate (pH 8.0) at 25° C.
^{c}Conjugates molecular weight was estimated from BCA as described previously¹⁴.
^{d}Number average molecular weight of cleaved pCBMA and dispersity index from GPC.
^{e}Estimated conjugates molecular weight from GPC.

TABLE 3
Characterization of bio-pro substrates

| | | Molecular | Biotin molecules |
|---|---|---|---|
| Sample | Dₕ^{5a} | weight (Da) | per protein^{b} |
| Aprotinin | 2.4 ± 0.4 | 6500 | 4 |
| Histone | 4.6 ± 0.2 | 21500 | 4 |
| HRP | 5.1 ± 0.7 | 44200 | 4 |

^{a}Hydrodynamic size of samples was determined by DLS.
^{b}Biotinylation of proteins was determined by fluorescamine assay.

TABLE 4
Biotin, biotin-protein and biotin-PEG binding kinetics to low-density avidin conjugates

| | | | | | Biotin- | Biotin- | Biotin- | Biotin- |
|---|---|---|---|---|---|---|---|---|
| | | Biotin- | Biotin- | Biotin- | PEG | PEG | PEG | PEG |
| | Biotin | aprotinin | histone | HRP | 550 Da | 5 kDa | 10 kDa | 30 kDa |
| Sample | k/s⁻¹ | k/s⁻¹ | k/s⁻¹ | k/s⁻¹ | k/s⁻¹ | k/s⁻¹ | k/s⁻¹ | k/s⁻¹ |
| Native avidin | 105.21 ± | 73.45 ± | 15.73 ± | 5.03 ± | 20.93 ± | 17.02 ± | 12.02 ± | 5.89 ± |
| | 13.01 | 2.13 | 1.34 | 0.77 | 1.22 | 2.31 | 0.36 | 0.42 |
| Avidin-pCBMA₅₆ | 0.763 ± | 0.565 ± | 0.282 ± | 0.261 ± | 0.738 ± | 0.359 ± | 0.157 ± | 0.156 ± |
| | 0.01 | 0.01 | 0.01 | 0.03 | 0.02 | 0.01 | 0.03 | 0.002 |
| Avidin-pCBMA₁₂₁ | 0.739 ± | 0.511 ± | 0.252 ± | 0.218 ± | 0.638 ± | 0.276 ± | 0.159 ± | 0.139 ± |
| | 0.03 | 0.02 | 0.02 | 0.02 | 0.03 | 0.01 | 0.04 | 0.004 |
| Avidin-pCBMA₁₇₀ | 0.682 ± | 0.493 ± | 0.245 ± | 0.209 ± | 0.62 ± | 0.275 ± | 0.149 ± | 0.136 ± |
| | 0.16 | 0.05 | 0.01 | 0.04 | 0.05 | 0.01 | 0.03 | 0.002 |
| Avidin-pCBMA₂₃₂ | 0.668 ± | 0.447 ± | 0.231 ± | 0.196 ± | 0.568 ± | 0.266 ± | 0.139 ± | 0.119 ± |
| | 0.05 | 0.12 | 0.01 | 0.03 | 0.09 | 0.02 | 0.02 | 0.001 |

Data were fit to single exponential functions using F(t) = F_{∞} − ΔFexp(−k_{obs}t) equation, where, k_{obs} is the observed first-order rate constant, F_{∞} is the final value of fluorescence and ΔF is the amplitude. In case of native avidin kinetics the data were fit to single exponential functions using F(t) = F_{∞} + ΔFexp(−k_{obs}t) equation, where, k_{obs} is the observed first-order rate constant, F_{∞} is the final value of fluorescence and ΔF is the amplitude.

TABLE 5
Characterization of bio-pro substrates.

| Sample | Dₕ^{5a} | Molecular weight (Da) |
|---|---|---|
| Bio-PEG 550 | 1.9 ± 0.8 | 550 |
| Bio-PEG 5K | 4.2 ± 0.5 | 5000 |
| Bio-PEG 10K | 6.1 ± 0.5 | 10000 |
| Bio-PEG 30K | 9.4 ± 0.9 | 30000 |

^{a}Hydrodynamic size of samples was determined by DLS.

TABLE 6
pCBMA characterization grown from free
single and double-headed initiators.

| Sample | Target DP | DP | Mₙ (Da) | M_{w} (Da) | PDI |
|---|---|---|---|---|---|
| Single-headed | 100 | 41 | 9300 | 12950 | 1.39 |
| Cleaved | | 40 | 9100 | 15560 | 1.27 |
| Double-headed | 200 (100) | 61 (30.5) | 14040 | 19760 | 1.4 |
| Cleaved | | 37 | 8580 | 11580 | 1.35 |

TABLE 8
Characterization of high-density avidin-pCBMA conjugates

| | | | Estimated | Cleaved | Estimated |
|---|---|---|---|---|---|
| | Polymerization | | conjugate | polymer^{d} | conjugate |
| | condition^{a} | | M_{w}^{c} (kDa) | Mₙ (kDa); | M_{w}^{e} (kDa) |
| Sample | [I]₀/[M]₀ | Dₕ^{b} | (BCA) | (M_{w}/Mₙ) | (GPC) |
| Avidin-pCBMA₅₈ | 1:100 | 25.1 ± 1.7 | 124 | 13.4 (1.6) | 316.2 |
| Avidin-pCBMA₁₀₉ | 1:150 | 30.8 ± 4.2 | 202 | 25.1 (1.8) | 648.5 |
| Avidin-pCBMA₁₅₂ | 1:200 | 34.4 ± 3.8 | 290 | 34.9 (1.8) | 895.5 |
| Avidin-pCBMA₁₈₂ | 1:250 | 38.8 ± 1.2 | 408 | 41.8 (1.8) | 1069.4 |

^{a}Seven initiators per avidin monomer, [I]₀/[Cu(II)Cl]₀/[NaAcs]₀[HMTETA]₀ = 1:10:1.2:10.
^{b}Hydrodynamic diameters (number distribution) of the avidin-pCBMA conjugates was measured using dynamic light scattering with sample concentration 1.0 mg/mL in 100 mM sodium phosphate (pH 8.0) at 25° C.
^{c}Conjugates molecular weight was estimated from BCA as described elsewhere (Murata et al., supra).
^{d}Number average molecular weight of cleaved pCBMA and dispersity index from GPC.
^{e}Estimated conjugate molecular weight from GPC.

TABLE 9
Biotin, biotin-protein and biotin-PEG binding kinetics to high-density avidin conjugates

| | | | | | Biotin- | Biotin- | Biotin- | Biotin- |
|---|---|---|---|---|---|---|---|---|
| | | Biotin- | Biotin- | Biotin- | PEG | PEG | PEG | PEG |
| | Biotin | aprotinin | histone | HRP | 550 Da | 5 kDa | 10 kDa | 30 kDa |
| Sample | k/s⁻¹ | k/s⁻¹ | k/s⁻¹ | k/s⁻¹ | k/s⁻¹ | k/s⁻¹ | k/s⁻¹ | k/s⁻¹ |
| Native avidin | 105.21 ± | 73.45 ± | 15.73 ± | 5.03 ± | 20.93 ± | 17.02 ± | 12.02 ± | 5.89 ± |
| | 13.01 | 2.13 | 1.34 | 0.77 | 1.22 | 2.31 | 0.36 | 0.42 |
| Avidin-pCBMA₅₈ | 0.345 ± | 0.056 ± | 0.039 ± | 0.028 ± | 0.082 ± | 0.046 ± | 0.025 ± | 0.018 ± |
| | 0.02 | 0.01 | 0.001 | 0.001 | 0.002 | 0.004 | 0.002 | 0.001 |
| Avidin-pCBMA₁₀₉ | 0.331 ± | 0.052 ± | 0.035 ± | 0.026 ± | 0.076 ± | 0.044 ± | 0.026 ± | 0.015 ± |
| | 0.02 | 0.002 | 0.001 | 0.001 | 0.003 | 0.002 | 0.003 | 0.002 |
| Avidin-pCBMA₁₅₂ | 0.31 ± | 0.047 ± | 0.034 ± | 0.025 ± | 0.073 ± | 0.042 ± | 0.024 ± | 0.016 ± |
| | 0.03 | 0.01 | 0.002 | 0.001 | 0.004 | 0.002 | 0.001 | 0.001 |
| Avidin-pCBMA₁₈₂ | 0.299 ± | 0.040 ± | 0.032 ± | 0.025 ± | 0.067 ± | 0.041 ± | 0.022 ± | 0.016 ± |
| | 0.02 | 0.008 | 0.002 | 0.001 | 0.001 | 0.002 | 0.002 | 0.001 |

Data were fit to single exponential functions using F(t) = F_{∞} − ΔFexp(−k_{obs}t) equation, where, k_{obs} is the observed first-order rate constant, F_{∞} is the final value of fluorescence and ΔF is the amplitude.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A polypeptide-polymer conjugate comprising:
a polypeptide,
one or more initiator molecules conjugated to the polypeptide, wherein each of said one or more initiator molecules comprises two or more atom transfer radical polymerization (ATRP) initiation groups, and
a polymer molecule conjugated to each of said ATRP initiation groups.

2. The polypeptide-polymer conjugate of claim 1, wherein the initiator molecule comprises two ATRP initiation groups.

3. The polypeptide-polymer conjugate of claim 2, wherein the initiator molecule is 4-(bis(2-(2-bromo-2-methylpropanamido)ethyl)amino)-4-oxobutyloyl-N-oxysuccinimide ester.

4. The polypeptide-polymer conjugate of claim 1, wherein the polymer is selected from the group consisting of poly(oligo(ethylene glycol) methacrylate) (pOEGMA), poly(carboxybetaine methacrylate) (pCBMA), copolymers of poly(oxyethylene)allylmethyldiether and maleic anhydride, copolymers of poly(oxyethylene)2-methyl-2-propenylmethyldiether and maleic anhydride, α-methoxy-poly(ethylene glycol) (MPEG), poly(polyethylene glycol monomethyl ether methacrylate) (PPEGMA), poly(2-dimethylaminoethyl methacrylate) (pDMAEMA), poly(sulfobetaine methacrylate) (pSBMA), poly(2-(methylsulfinyl)ethyl acrylate) (pMSEA), poly(N,N-dimethylaminoethyl methacrylate), poly(quaternary ammonium ethyl methacrylate), poly(hydroxyethyl)methacrylate, 2-azidoethyl methacrylate, and epoxy methacrylate.

5. The polypeptide-polymer conjugate of claim 1, wherein the polypeptide is an enzyme.

6. The polypeptide-polymer conjugate of claim 5, wherein the enzyme is an esterase, lipase, organophosphate hydrolase, aminase, oxidoreductase, hydrogenase, lysozyme, transaminase, asparaginase, protease, or uricase.

7. A method for generating a polypeptide-polymer conjugate, said method comprising:
coupling one or more initiator molecules to a polypeptide to generate a polypeptide-initiator complex, wherein each of said one or more initiator molecules comprises two or more ATRP initiation groups; and
growing, via controlled radical polymerization, a polymer molecule from each of said two or more ATRP initiation groups, thus generating a polypeptide-polymer conjugate.

8. The method of claim 7, wherein the initiator molecule comprises two ATRP initiation groups.

9. The method of claim 7, wherein the ATRP initiation groups are alkyl bromide or alkyl chloride groups.

10. The method of claim 7, wherein the initiator molecule is 4-(bis(2-(2-bromo-2-methylpropanamido)ethyl)amino)-4-oxobutyloyl-N-oxysuccinimide ester.

11. The method of claim 7, wherein the polymer is selected from the group consisting of pOEGMA, pCBMA, copolymers of poly(oxyethylene)allylmethyldiether and maleic anhydride, copolymers of poly(oxyethylene)2-methyl-2-propenylmethyldiether and maleic anhydride, MPEG, PPEGMA, pDMAEMA, pSBMA, pMSEA, poly(N,N-dimethylaminoethyl methacrylate), poly(quaternary ammonium ethyl methacrylate), poly(hydroxyethyl)methacrylate), 2-azidoethyl methacrylate, and epoxy methacrylate.

12. The method of claim 7, wherein the polypeptide is an enzyme.

13. The method of claim 12, wherein the enzyme is an esterase, lipase, organophosphate hydrolase, aminase, oxidoreductase, hydrogenase, lysozyme, transaminase, asparaginase, protease, or uricase.

14. The method of claim 7, wherein the controlled radical polymerization is atom transfer radical polymerization.

15. A polypeptide-polymer conjugate obtained by coupling one or more initiator molecules to a polypeptide to generate a polypeptide-initiator complex, wherein each of said one or more initiator molecules comprises two or more ATRP initiation groups; and growing, via controlled radical polymerization, a polymer molecule from each of said two or more ATRP initiation groups, thus generating a polypeptide-polymer conjugate.

16. A conjugate comprising a polypeptide having one or more initiator molecules coupled thereto, wherein each initiator molecule comprises two or more ATRP initiation groups.

17. The conjugate of claim 16, wherein the initiator molecule is 4-(bis(2-(2-bromo-2-methylpropanamido)ethyl)amino)-4-oxobutyloyl-N-oxysuccinimide ester.
